(19) United States
(12) Patent Application Publication
(10) Pub. No.: US 2023/0096410 A1
(43) Pub. Date: Mar. 30, 2023

(54) Innate Immunity Killer Cells Targeting PSMA Positive Tumor Cells

(71) Applicant: Sorrento Therapeutics, Inc., San Diego, CA (US)

(72) Inventors: Yanwen Fu, San Diego, CA (US); Reyna Lim, San Diego, CA (US); Daniel Lee, San Diego, CA (US); Matthew Buschman, San Diego, CA (US); Tong Zhu, San Diego, CA (US); Alisher B. Khasanov, San Diego, CA (US)

(73) Assignee: Sorrento Therapeutics, Inc., San Diego, CA (US)

(21) Appl. No.: 17/908,814

(22) PCT Filed: Mar. 5, 2021
(86) PCT No.: PCT/US2021/021204

§ 371 Date: Sep. 1, 2022

Related U.S. Application Data
(60) Provisional application No. 62/986,392, filed on Mar. 6, 2020.

(51) Int. Cl. A61K 35/17; A61P 35/00; A61K 31/164

(52) U.S. Cl. CPC A61K 35/17; A61P 35/00; A61K 31/164

(57) ABSTRACT

The present disclosure provides an innate immunity cell such as a gamma delta T (gdT) cell, Natural Killer (NK) cell, or macrophage having 2-[3-(1,3-dicarboxypropyl)ureido] pentanedioic acid (DUPA) chemically conjugated to the cell surface. The DUPA-conjugated cells provided herein demonstrate increased cytotoxicity toward cancer cells expressing PSMA. DUPA-conjugated cells can be primary cells or cells of a cell line. Also provided are methods of conjugating DUPA to the surface of NK cells, gamma delta T (gdT) cells, or macrophages and methods of treating cancer using DUPA-conjugated NK cells, gamma delta T (gdT) cells, or macrophages.

This application claims the benefit of priority of U.S. Provisional Patent Application No. 62/986,392, filed Mar. 6, 2020, which is incorporated by reference herein in its entirety for all purposes.

### TECHNICAL FIELD

The invention relates to adoptive cell therapy using cells of the innate immune system, such as Natural Killer (NK) cells, Macrophages, and Gamma Delta T cells. More particularly, the invention provides NK cells, Macrophages, and Gamma Delta T cells having the small molecule DUPA chemically conjugated to the cell surface.

### BACKGROUND OF THE INVENTION

Natural Killer (NK) cells are specialized effectors of the innate immune system that rapidly respond to and attack virus-infected cells and poorly differentiated tumor cells in an antigen-independent manner (Ames et al. (2015) J. Immunol. 195:4010-019). NK cells are characterized as lymphocytes that express CD56 but do not express CD3 or CD19. In humans, two main subsets of NK cells are characterized by their expression levels of CD56 and CD16. A CD16^{dim}CD56^{bright} subpopulation of NK cells predominates in secondary lymphoid tissues and has a regulatory function, secreting cytokines that activate cytotoxic NK cells and T cells. The other major subpopulation, comprising CD16^{bright}CD56^{dim} NK cells, predominates in the peripheral blood and exhibits cytotoxicity toward virus-infected host cells as well as some tumors and cancer stem cells. Cytolysis of target cells by NK cells occurs by the release of perform and granzyme B which are able to induce both necrotic and apoptotic cell death (Jewett et al. (2020) Mol. Ther. Oncolytics 16:41-52). The cytotoxicity of NK cells toward target cells does not require pre-exposure to infected or transformed cells or presentation of non-self antigens in the context of MHC molecules.

Cytotoxicity of NK cells toward normal healthy cells is prevented by means of the inhibitory killer Ig-like receptors (KIRs) including KIR2DL1, KIR2D2/L3, KIR3DL1, and KIR3DL as well as CD94/NKG2A that are expressed by the NK cells. The interaction of any of these receptors with HLA molecules on the surface of potential target cells inhibits the cytolytic program of NK cells so that only cells having reduced or absent HLA expression as the result of viral infection or tumor transformation are attacked (Gras Navarro et al. (2015) Front. Immunol. 6: article 202, 1-18).

Cytotoxic NK cells are positively regulated by the interactions of other cell surface receptors expressed by NK cells with binding partners present on targets. For example, toll-like receptors (TLRs, e.g., TLR2, TLR3, TLRS, TLR7/8, TLR9) bind bacterial and viral ligands, and natural cytotoxicity receptors (NCRs, e.g., NKp30, NKp44, NKp46) bind ligands expressed on some tumor cells and on virally infected cells. Other regulatory signals are transmitted by cytokines or chemokines, such as for example IL-12, which is known to activate NK cells (Sivori et al. (2014) Front. Immunol. 5: article 105, 1-10.

NK cells have also been demonstrated to express checkpoint inhibitors (CIs), including PD-1, CTLA-4, TIM-3, LAG-3, TIGIT, and CD96. Interaction of the checkpoint inhibitors with their ligands negatively regulates the cytolytic activity of NK cells (Lanuza et al. (2020) Front. Immunol. 5: article 3010, 1-11).

Because NK cells exhibit cytotoxicity to many tumors, adoptive cell immunotherapy to treat cancer has been attempted using both autologous and haploidentical (allogeneic) NK cells (Becker et al. 2016 Cancer Immunol Immunother 65:477-484; Gras Navarro et al. 2015). Treatments using autologous cells rely on harvesting the patient's blood cells, enriching for NK cells, and expanding and activating the NK cells in culture to enhance their tumor-killing ability. Difficulties in this approach include overcoming negative regulation by self-HLA molecules on the tumor and maintaining the re-introduced NK cells in an activated state. The use of haplotype-matched allogeneic NK cells has been more successful, but difficulties remain in achieving adequate NK cell expansion, poor persistence of the allogeneic cells in the patient, and immunosuppressive mechanisms of the tumors.

Macrophages are monocyte-derived cells that also participate in innate immunity. These cells are found throughout the body where they survey tissues for pathogens or cellular debris which are then phagocytosed. Macrophages may also participate in the adaptive immune system by presenting antigens to T cells.

Gamma delta T cells (gdT cells) are another type of immune cell that participate in both innate and adaptive immunity. These T cells do not require MHC antigen presentation for activation but can recognize pathogen-specific molecules. Gamma delta T cells when activated have cytolytic activity and can also regulate other immune cells by secreting cytokines that may stimulate or suppress the activity of macrophages, dendritic cells, NK cells, or CD8+ T cells.

Prostate cancer is the second-most common cancer in men, and the second leading cause of cancer deaths in men in the United States. In 2020, it is estimated that there will be about 191,930 new cases of prostate cancer and 33,330 deaths due to prostate cancer in the US (American Cancer Society). Current treatments for aggressive prostate cancer include surgery, hormone therapy, radiotherapy, and chemotherapy.

The prostate-specific membrane antigen (PSMA) or glutamate carboxypeptidase II, also known as N-acetyl-L-aspartyl-L-glutamate peptidase or NAAG peptidase is overexpressed on prostate cancer cells, including metastases at distant sites (O'Keefe et al. (2018) J. Nucl. Med. 59:-1007-1013). The small molecule 2-[3-(1,3-dicarboxypropyl)ureido]pentanedioic acid (DUPA) specifically binds PSMA; conjugates of fluorophores, radionuclides, and chemotoxines with DUPA bind PSMA with a Kd in the nanomolar range (Kularatne et al. 2009 Mol Pharm. 6:780-789). DUPA has been conjugated to labeling moieties such as ⁶⁸Ga, chromophores, and fluorophores for imaging of tumors and detection of metastases as well as for flow cytometry and other applications (Kulartne et al. (2009) Mol Pharm. 6:790-800; U.S. Pat. No. 8,685,752). DUPA has also been conjugated to cytotoxic agents such as tubulysin B and radionucleides such as ¹⁸F, ¹³¹I, ^{99m}Tc, and ¹⁷⁷Lu for targeted delivery to prostate cancer cells (U.S. Pat. No. 8,907,058, Kularatne et al. 2009 Mol Pharm. 6:780-789; Kularatne et al. 2009 Mol Pharm. 6:790-800; Kularatne et al. 2010 J. Med Chem 53:7767-77; Afshar-Oromieh et al. (2016) J Nucl. Med. 57, Suppl 3:79S-89S).

### SUMMARY

In a first aspect, provided herein is a cell of the innate immune system that has the small molecule 2-[3-(1,3-dicarboxypropyl)ureido]pentanedioic acid (DUPA) chemically conjugated to the cell surface. The DUPA-conjugated cell exhibits increased cytotoxicity toward tumor cells expressing PSMA as compared to a substantially identical cell that does not have DUPA conjugated to the cell surface. The cell may be, for example, an NK cell, a macrophage, or a gamma delta T (gdT) cell. The cell can be a primary cell or a cell of a cell line. In various embodiments the cell is a human cell.

DUPA can be conjugated to the surface of an NK cell, macrophage, or gdT cell via a linker, where the linker can include a functional group that can be used to covalently attach the DUPA compound to the cell surface. Functional groups used for conjugation to the cell surface include for example groups reactive with sulfhydryls or amines. For example, a linker used to couple DUPA to the surface of a cell can include an amine-reactive group such as but not limited to N-hydroxysuccinimide (NHS), pentafluorophenyl, tetrafluorophenyl, nitrophenyl, isocyanate, tetrafluorobenzenesulfonate, isothiocyanate, or sulfonylchloride. In exemplary embodiments DUPA is conjugated to the cell surface via a linker that includes an NHS functional group.

A linker that connects DUPA to the surface of an innate immunity cell, such as an NK cell, macrophage, or gamma delta T cell, can also include a spacer attached to the functional group, which can be any chemical spacer that is sufficiently soluble in cell buffers and media and non-toxic to the cells. For example, a linker can include, as nonlimiting examples, any of the following: an amino acid, a dipeptide, a tripeptide, polyglycine, p-aminobenzyl (PAB), a sugar, piperazine, piperidine, a triazoyl, (CH₂)ₙ-, —(CH₂CH₂O)ₙ-, —(C═O)—, —CH₂(C═O)—, —(C═O)—CH₂—, —(C═O)CH₂CH₂O)—, —CH₂CH₂NH—, —(CH₂CH₂O)ₙ—CH₂CH₂NH—, —(C═O)CH₂CH₂(C═O), —(C═O)CH₂CH₂O(CH₂CH₂O)ₙCH₂CH₂NH(C═O)—, —(C═O)CH₂CH₂O(CH₂CH₂O)ₙCH₂CH₂NH—, and —(C═O)CH₂CH₂OCH₂CH₂OCH₂CH₂—, where n can be, independently, 1-30. A linker used for attaching DUPA to a cell surface can include any combinations of the foregoing groups or moieties, optionally in combination with other chemical groups or moieties in the linker. In some embodiments, a linker can include a spacer that has a length of at least 25 Angstroms, at least 50 Angstroms, at least 75 Angstroms, or at least 100 Angstroms, or, for example, can have a linker with a chain length of at least 16 atoms, at least 32 atoms, at least 50 atoms, at least 65 atoms, at least 70 atoms, or at least 75 atoms. A spacer can be of any length, but in some embodiments a spacer of a linker of a DUPA compound that connects DUPA to the functional group used for conjugation to cells can in some embodiments have a length of from about 50 angstroms to about 400 angstroms or greater, for example from about 50 angstroms to about 300 angstroms, or from about 100 Angstroms to about 400 Angstroms, from about 100 Angstroms to about 350 Angstroms, from about 50 Angstroms to about 250 Angstroms, from about 80 Angstroms to about 250 Angstroms, from about 90 Angstroms to about 250 Angstroms, from about 100 angstroms to about 250 Angstroms, from about 80 Angstroms to about 150 Angstroms, or from about 100 Angstroms to about 150 Angstroms.

In a further aspect, disclosed herein is a population of innate immune system cells have DUPA conjugated to the cell surface as provided herein. The population of DUPA-conjugated innate immune cells may be, for example, NK cells, gamma delta T cells, or macrophages. The population of cells can be a population of primary cells or can be a population of cells of a cell line. The population may be a population that has been activated and/or selectively enriched for a particular cell type or expanded, for example using one or more cell-binding ligands or antibodies and/or cytokines. The population of cells may be a population of cells of a cell line, for example, that has been irradiated such that the population is viable but non-dividing. The population may be provided in a cell medium, or in PBS or another buffer, where the medium or buffer can optionally include a cryoprotectant such as glycerol.

Also included is a pharmaceutical composition that comprises a population of DUPA-conjugated innate immune system cells as provided herein for administration to a patient. The cells can be provided in a culture medium or can be provided in PBS or another buffer. The composition can optionally be frozen. The pharmaceutical composition can be provided in a bag, vial, tube, or other container for administration of a single dose or multiple doses.

Also provided are methods of treating a subject having a PSMA-positive tumor, where the methods include administering to the subject an effective amount of a population of DUPA-conjugated cells as provided herein. In various embodiments the cells can be DUPA-conjugated NK cells, DUPA-conjugated gamma delta T cells, or DUPA-conjugated macrophages. The cells can be administered by any suitable means, for example, by injection or infusion, in one or multiple doses. Where multiple dosings are employed, the doses of cells can be separated by hours, days, weeks, or months. The tumor can be a solid tumor, and in exemplary embodiments is prostate cancer.

In some embodiments, a population of macrophages is provided where the macrophages have DUPA conjugated to the cell surface. The macrophages can be derived from monocytes isolated from blood or can be tissue-derived primary macrophages. Alternatively the population of macrophages can be cells of a cell line, such as a human macrophage cell line. Also provided is a pharmaceutical composition that includes a population of macrophages having DUPA conjugated to the cell surface. The pharmaceutical composition can include macrophages provided in a culture medium or in PBS or another cell-compatible buffer, where the culture medium or buffer can optionally include a cryoprotectant, such as, for example, glycerol or

DMSO. The pharmaceutical composition can be provided in a vial, bag, tube, or other container for administration of a single dose or multiple doses and can optionally be provided frozen.

Also provided are methods of treating a treating a subject having a PSMA-positive tumor, comprising administering to the patient an effective amount of a population of DUPA-conjugated macrophages as provided herein. The cells can be administered, for example, by injection or infusion, in one or multiple doses. The tumor can be a solid tumor, and in exemplary embodiments is prostate cancer.

In further embodiments, a population of gdT cells is provided where the gdT cells have DUPA conjugated to the cell surface. The gdT cells can be primary cells, for example, isolated from PBMCs, umbilical cord, or placenta, or can be cells of a gdT cell line, such as a human gdT cell line. Further provided is a pharmaceutical composition that includes a population of gdT cells having DUPA conjugated to the cell surface. The pharmaceutical composition can include gdT cells provided in a culture medium or in PBS or another cell-compatible buffer, where the culture medium or buffer can optionally include a cryoprotectant, such as, for example, glycerol or DMSO. The pharmaceutical composition can be provided in a vial, bag, tube, or other container for administration of a single dose or multiple doses and can optionally be provided frozen.

Also provided are methods of treating a treating a subject having a PSMA-positive tumor, comprising administering to the patient an effective amount of a population of DUPA-conjugated gdT cells as provided herein. The cells can be administered, for example, by injection or infusion, in one or multiple doses. The tumor can be a solid tumor, and in exemplary embodiments is prostate cancer.

In additional embodiments, a population of NK cells is provided where the NK cells have DUPA conjugated to the cell surface. The NK cells can be primary cells, for example, isolated from PBMCs, umbilical cord, or placenta, or can be cells of a NK cell line, such as a human NK cell line, e.g., can be KHYG cells. Further provided is a pharmaceutical composition that includes a population of NK cells having DUPA conjugated to the cell surface. The pharmaceutical composition can include NK cells provided in a culture medium or in PBS or another cell-compatible buffer, where the culture medium or buffer can optionally include a cryoprotectant, such as, for example, glycerol or DMSO. The pharmaceutical composition can be provided in a vial, bag, tube, or other container for administration of a single dose or multiple doses and can optionally be provided frozen.

Also provided are methods of treating a treating a subject having a PSMA-positive tumor, comprising administering to the patient an effective amount of a population of DUPA-conjugated NK cells as provided herein. The cells can be administered, for example, by injection or infusion, in one or multiple doses. The tumor can be a solid tumor, and in exemplary embodiments is prostate cancer.

Another aspect of the disclosure is a method of producing a DUPA-conjugated cell population comprising conjugating a compound comprising DUPA to the surface of cells. The cells can be cells of the innate immune system, for example, NK cells, macrophages, or gdT cells. The DUPA compound can be a compound that comprises DUPA linked to a functional group, such as a group that reacts with sulfhydryls or amines. The conjugation conditions can be any that result in conjugation of the functional group with a corresponding reactive group on the cell surface and compatible with the viability and functionality of the cells. In certain exemplary embodiments, the method can include contacting a compound that comprises DUPA and a linker that includes a functional group with a population of cells and incubating the cells with the DUPA compound for a period of time and at a temperature that results in conjugation of the DUPA compound to the cell surface. In some embodiments the method can include providing a population of NK cells by isolation, enrichment, and/or selective expansion or a combination thereof from a sample derived from one or more donors. The sample may be from peripheral blood (e.g., PBMCs), cord blood, or placenta, as nonlimiting examples. In some embodiments the method can include providing a population of gdT cells by isolation, enrichment, and/or selective expansion or a combination thereof from a sample derived from one or more donors. The sample may be from peripheral blood (e.g., PBMCs), cord blood, or placenta, for example. In further embodiments the method can include providing a population of NK cells, macrophages, or gdT cells by isolation, enrichment, and/or selective expansion or a combination thereof from a sample derived from one or more donors.

### BRIEF DESCRIPTION OF THE DRAWINGS

FIG. 1 provides the structures of small molecule compounds DUPA-BisPhe-L1, DUPA-BisPhe-L2, Sulfo-NHS-LC Biotin, and DBCO. DUPA-BisPhe-L1, DUPA-BisPhe-L2, and Sulfo-NHS-LC Biotin are attached to linkers that include the functional group NHS or Sulfo-NHS group (biotin).

FIG. 2 provides graphs of the percent cytolysis of target cells by non-conjugated KHYG NK effector cells and KHYG NK effector cells conjugated with either 200μM or 353μM DUPA-BisPhe-L1 over time in electrical impedance-based real-time XCELLIGENCE® cytotoxicity assays: A) curve of cytolysis at 10:1 effector to target ratios, where targets were LNCaP (PSMA+) cells plated at time 0 and effectors were added at 24 hours; B) bar chart of cytolysis of LNCaP target cells at 10:1 effector to target ratios at 20, 24, and 30 hours after the addition of target cells; C) curve of cytolysis of LNCaP target cells at 5:1 effector to target ratios, where targets were plated at time 0 and effectors were added at 24 hours; D) bar chart of cytolysis of LNCaP target cells at 5:1 effector to target ratios at 24, 41, and 72 hours after the addition of target cells; E) curve of cytolysis of PC3 (PSMA-) target cells at 10:1 effector to target ratio.

FIG. 3 provides graphs of the percent cytolysis over time of LNCaP (PSMA+) target cells by non-conjugated KHYG NK effector cells and KHYG NK effector cells conjugated with either 50 μM DUPA-BisPhe-L1 (“DUPA”), 200 μM DUPA-BisPhe-L1, or 200 μM of the DBCO compound of FIG. 2C (“DBCO”)in XCELLIGENCE® cytotoxicity assays: A) curve of cytolysis at 10:1 effector to target ratios; B) curve of cytolysis at 5:1 effector to target ratios; C) curve of cytolysis at 2.5:1 effector to target ratios; D) curve of cytolysis at 1.25:1 effector to target ratios; E) curve of cytolysis at 0.625:1 effector to target ratios. In all graphs, target cells were plated at time 0 and effectors were added at 24 hours.

FIG. 4 provides bar graphs providing the percent cytolysis at various timepoints of the assays depicted in FIG. 3. A) shows the percent target cell cytolysis at 6 hours after KHYG effector cell addition; B) shows the percent target cell cytolysis at 24 hours after KHYG effector cell addition.

FIG. 5 provides graphs of the percent cytolysis over time of PC3 (PSMA-) target cells by non-conjugated KHYG effector cells and KHYG effector cells conjugated with either 50 μM DUPA-BisPhe-L1 (“DUPA”), 200 μM DUPA-BisPhe-L1, or 200 μM of the DUPA compound of FIG. 2A (“DBCO”) in XCELLIGENCE® cytotoxicity assays: A) curve of cytolysis at 10:1 effector to target ratios; B) curve of cytolysis at 5:1 effector to target ratios; C) curve of cytolysis at 2.5:1 effector to target ratios; D) curve of cytolysis at 1.25:1 effector to target ratios; E) curve of cytolysis at 0.625:1 effector to target ratios. Target cells were plated at time 0 and effectors were added at 24 hours.

FIG. 6 provides graphs of the percent cytolysis of LNCaP (PSMA+) target cells by non-conjugated KHYG effector cells and KHYG effector cells conjugated with either 200 μM DUPA-BisPhe-L1 (DUPA-KHYG), or 200 μM DUPA-BisPhe-L2 ((Long) DUPA-PEG6-KHYG) over time in XCELLIGENCE® cytotoxicity assays: A) curve of cytolysis at 10:1 effector to target ratios; B) curve of cytolysis at 5:1 effector to target ratios; C) curve of cytolysis at 2.5:1 effector to target ratios; D) curve of cytolysis at 1.25:1 effector to target ratios; E) curve of cytolysis at 0.625:1 effector to target ratios. Target cells were plated at time 0 and effectors were added at 24 hours.

FIG. 7 provides graphs of the percent cytolysis of PC3 (PSMA-) target cells by non-conjugated KHYG effector cells and KHYG effector cells conjugated with either 200 μM DUPA-BisPhe-L1 (DUPA-KHYG) or 200 μM DUPA-BisPhe-L2 ((Long)DUPA-PEG6-KHYG) over time in XCELLIGENCE® cytotoxicity assays: A) curve of cytolysis at 10:1 effector to target ratios; B) curve of cytolysis at 5:1 effector to target ratios; C) curve of cytolysis at 2.5:1 effector to target ratios; D) curve of cytolysis at 1.25:1 effector to target ratios; E) curve of cytolysis at 0.625:1 effector to target ratios. Target cells were plated at time 0 and effectors were added at 24 hours.

FIG. 8 provides bar graphs providing cytolysis at various timepoints of the assays depicted in FIG. 7: A) percent target cell cytolysis at 6 hours after KHYG effector cell addition; B) percent target cell cytolysis at 24 hours after KHYG effector cell addition.

FIG. 9 provides graphs of the percent cytolysis of LNCaP (PSMA+) target cells by non-conjugated KHYG effector cells and KHYG effector cells conjugated with either 50 μM DUPA-BisPhe-L1 (“50 μM DUPA-KHYG”), 200 μM DUPA-BisPhe-L1 (“200 μM DUPA-KHYG”), 50 μM DUPA-BisPhe-L2 (“50 μM DUPA-PEG6-KHYG”), or 200 μM DUPA-BisPhe-L2 ((“200 μM DUPA-PEG6-KHYG”) over time in XCELLIGENCE® cytotoxicity assays: A) curve of cytolysis at 10:1 effector to target ratios; B) curve of cytolysis at 5:1 effector to target ratios; C) curve of cytolysis at 2.5:1 effector to target ratios; D) curve of cytolysis at 1.25:1 effector to target ratios; E) curve of cytolysis at 0.625:1 effector to target ratios.

FIG. 10 provides graphs of the percent cytolysis of PC3 (PSMA−) target cells by KHYG effector cells at 10:1, 3.3:1, 1.1:1, 0.37:1, and 0.123:1 effector to target ratios; A) curve of cytolysis using non-conjugated KHYG cells as effectors; B) curve of cytolysis using KHYG cells conjugated with 200 μM DUPA-BisPhe-L2 ((“200 μM DUPA-PEG6-KHYG”) as effectors.

FIG. 11 provides bar graphs providing the percent cytolysis at various timepoints of the assays depicted in FIG. 10. A) shows the percent target cell cytolysis at 6 hours after KHYG effector cell addition; B) shows the percent target cell cytolysis at 24 hours after KHYG effector cell addition.

FIG. 12 provides the results of flow cytometry analysis performed to demonstrate the efficiency of conjugation of small molecules to cells. A) gdT cells conjugated to DUPA and then sequentially bound with PSMA-Fc and APC-anti-human IgG, showing that 98.5% of the cells reacted with DUPA were DUPA-positive as assessed by flow cytometry, and B) gdT cells conjugated to biotin and then sequentially bound with FITC-streptavidin, showing that 99.5% of the cells reacted with biotin were biotin-positive.

FIG. 13 provides graphs of the percent cytolysis of LNCaP (PSMA+) target cells by non-conjugated gdT cells and gdT cells conjugated with either DUPA-BisPhe-L1 (gdT-DUPA-L1), or DUPA-BisPhe-L2 (gdT-DUPA-L2) over time in XCELLIGENCE® cytotoxicity assays: A) curve of cytolysis at 3:1 effector to target ratios; B) curve of cytolysis at 1:1 effector to target ratios; C) curve of cytolysis at 0.3:1 effector to target ratios; and D) curve of cytolysis at 0.1:1 effector to target ratios. Target cells were plated at time 0 and effectors were added approximately 25 hours later.

FIG. 14 provides bar graphs of the % cytolysis at various time points based on the impedance assays shown in FIG. 13. A) Per cent cytolysis of LNCaP (PSMA+) target cells 2 hours after addition of effectors, B) Per cent cytolysis of LNCaP (PSMA+) target cells 6 hours after addition of effectors, C) Per cent cytolysis of LNCaP (PSMA+) target cells 24 hours after addition of effectors.

FIG. 15 provides graphs of the percent cytolysis of PC3 (PSMA−) target cells in assays with non-conjugated gdT cells and gdT cells conjugated with either DUPA-BisPhe-L1 (gdT-DUPA-L1), or DUPA-BisPhe-L2 (gdT-DUPA-L2) as effectors over time in

XCELLIGENCE® cytotoxicity assays: A) curve of cytolysis at 3:1 effector to target ratios; B) curve of cytolysis at 1:1 effector to target ratios; C) curve of cytolysis at 0.3:1 effector to target ratios; and D) curve of cytolysis at 0.1:1 effector to target ratios. Target cells were plated at time 0 and effectors were added approximately 25 hours later.

FIG. 16 provides bar graphs of the % cytolysis at various time points based on the impedance assays shown in FIG. 15. A) Per cent cytolysis of PC3 (PSMA−) target cells 2 hours after addition of effectors, B) Per cent cytolysis of target cells 6 hours after addition of effectors to LNCaP target cells, C) Per cent cytolysis of target cells 24 hours after addition of effectors to LNCaP target cells.

FIG. 17 provides the results of XCELLIGENCE® impedance-based cytotoxicity assays using PSMA-positive LNCaP target cells and the results of cytotoxicity assays using PSMA-negative PC3 target cells in the same graphs. Effectors were assayed separately with both PSMA-positive LNCaP target cells and PSMA-negative PC3 target cells. Target cells were plated at time 0 and effectors were added approximately 25 hours later. A) provides curves of cytolysis at 3:1 effector to target ratios, where the effectors are DUPA-BisPhe-L1-conjugated gdT cells (gdT-DUPA-L1), DUPA-BisPhe-L2-conjugated gdT cells (gdT-DUPA-L2), or, as controls, unconjugated gdT cells or gdT cells conjugated to biotin. B) provides curves of cytolysis at 1:1 effector to target ratios, where the effectors are DUPA-BisPhe-L1-conjugated gdT cells (gdT-DUPA-L1), DUPA-BisPhe-L2-conjugated gdT cells (gdT-DUPA-L2), or, as controls, unconjugated gdT cells or gdT cells conjugated to biotin. C) provides curves of cytolysis at 0.3:1 effector to target ratios, where the effectors are DUPA-BisPhe-L1-conjugated gdT cells (gdT-DUPA-L1), DUPA-BisPhe-L2-conjugated gdT cells (gdT-DUPA-L2), or, as controls, unconjugated gdT cells or gdT cells conjugated to biotin. D) provides curves of cytolysis at 0.1:1 effector to target ratios, where the effectors are DUPA-BisPhe-L1-conjugated gdT cells (gdT-DUPA-L1), DUPA-BisPhe-L2-conjugated gdT cells (gdT-DUPA-L2), or, as controls, unconjugated gdT cells or gdT cells conjugated to biotin.

FIG. 18 provides bar graphs of the % cytolysis at various time points based on the impedance assays shown in FIG. 17. A) Per cent cytolysis of PC3 (PSMA−) and LNCaP (PSMA+) target cells 2 hours after addition of effectors, B) Per cent cytolysis of PC3 (PSMA−) and LNCaP (PSMA+) target cells 6 hours after addition of effectors, and C) Per cent cytolysis of PC3 (PSMA−) and LNCaP (PSMA+) target cells 24 hours after addition of effectors.

### DETAILED DESCRIPTION OF THE INVENTION

Throughout this application, including the Background section and Examples, various publications, patents, and/or patent applications are referenced. The disclosures of the publications, patents and/or patent applications are hereby incorporated by reference in their entireties into this application in order to more fully describe the state of the art to which this disclosure pertains.

Unless specifically indicated otherwise, all technical and scientific terms used herein have the same meaning as commonly understood by those of ordinary skill in the art. In addition, any method or material similar or equivalent to a method or material described herein can be used in the practice of the present disclosure.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “primary cell” refers to a cell isolated directly from a multicellular organism. Primary cells typically have undergone very few population doublings and are therefore more representative of the main functional component of the tissue from which they derived in comparison to continuous (tumor or artificially immortalized) cell lines. In some cases, primary cells cannot divide indefinitely and thus cannot be cultured for long periods of me in vitro.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

The term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Administration by injection can be, as nonlimiting examples, intravenous, intraperitoneal, intramuscular, intratumoral, or peritumoral. Other modes of delivery include, but are not limited to, the use of intravenous infusion or implantation of a matrix or polymer comprising the conjugated cells.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., DUPA-conjugated NK cells) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other agents, timing of administration, and the physical delivery system in which it is carried.

The term “pharmaceutically acceptable carrier” refers to a substance that aids the administration of an agent (e.g., DUPA-conjugated NK cells) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, and the like. One of skill in the art will recognize that other pharmaceutical carriers are useful in the present invention.

Headings are solely for the convenience of the reader, and do not limit the invention or its embodiments.

### Cells Having DUPA Conjugated to the Cell Surface

Cells that exhibit HLA-independent cytotoxicity toward bacteria, virus-infected cells, and tumor cells that express abnormal molecules can be considered cells of the innate immune system and can be used in the compositions and methods provided herein. Exemplary cells considered for conjugation with DUPA include macrophages, Natural Killer (NK) cells, and gamma delta T (gdT) cells. Because these cells are activated by HLA-independent mechanisms, they are unlikely to generate graft-versus-host disease and cytokine release syndrome when delivered to a subject.

DUPA (2-[3-(1,3-dicarboxypropyl)ureido]pentanedioic acid) is a small molecule that specifically binds PSMA; binding assays using DUPA conjugated to a labeled moiety have provided a Kd for binding to PSMA of 14 nM (Kularatne et al. (2009) Mol Pharm. 6:780-789). DUPA can be conjugated to the surface of a cell, such as an NK cell, macrophage, or gdT cell, by means of a functional group attached to DUPA via a spacer. In various preferred embodiments, a DUPA compound is conjugated directly to the cell surface, where a “DUPA compound” refers to a compound that comprises DUPA and a linker, where the linker comprises a spacer and a functional group. In various examples, the functional group of the linker is a group that reacts with amines or sulfhydryls that may be present on the cell surface, such as exposed lysines or cysteines of cell membrane proteins.

Nonlimiting examples of functional groups that can react with sulfhydryls include, without limitation, maleimide, pyridyldithio, bromoacetyl, iodoacetyl, bromobenzyl, iodobenzyl, and 4-(cyanoethynyl)benzoyl. Functional groups used for conjugation to cell surface lysines include, as nonlimiting examples, N-hydroxysuccinimide (NHS), pentafluorophenyl, tetrafluorophenyl, tetrafluorobenzenesulfonate, nitrophenyl, isocyanate, isothiocyanate, and sulfonylchloride. In exemplary embodiments DUPA is conjugated to the surface of an NK cell, macrophage, or gdT cell, via lysine-reactive functional group such as NHS that is attached to DUPA via a spacer.

In some exemplary embodiments, NK cells, macrophages, or gdT cells as provided herein have DUPA conjugated to the cell surface via a linker that includes a functional group, e.g., such as but not limited to N-hydroxysuccinimide (NHS), that allows conjugation of DUPA to exposed lysine residues on the cell surface.

Linkers that include functional group for conjugation to the cell surface preferably also include a spacer between the functional group and the DUPA moiety. A spacer can be any composition or length, and in various exemplary embodiments can include, without limitation, any of the following, including any combinations of one or more of the following: an amino acid, a dipeptide, a tripeptide, polyglycine, p-aminobenzyl (PAB), a sugar, piperazine, piperidine, a triazoyl, (CH₂)ₙ-, —(CH₂CH₂O)ₙ-, —(C═O)—, —CH₂(C═O)—, —(C═O)—CH₂—, —(C═O)CH₂CH₂O)—, —CH₂CH₂NH—, —(CH₂CH₂O)ₙ—CH₂CH₂NH—, —(C═O)CH₂CH₂(C═O), —(C═O)CH₂CH₂O(CH₂CH₂O)ₙCH₂CH₂NH(C═O)—, —(C═O)CH₂CH₂O(CH₂CH₂O)ₙCH₂CH₂NH—, and —(C═O)CH₂CH₂OCH₂CH₂OCH₂CH₂—, where n can be, independently, 1-30. A linker used for attaching DUPA to a cell surface can include any combinations of the foregoing groups or moieties, optionally in combination with other chemical groups or moieties in the linker. In some embodiments the spacer will include at least one of (CH₂)ₙ-, —(C═O)—, —(CH₂CH₂O)ₙ-, and —CH₂(C═O)—. In some embodiments the spacer does not include a cleavage site for a protease or peptidase, for example, does not include a cathepsin B cleavage site.

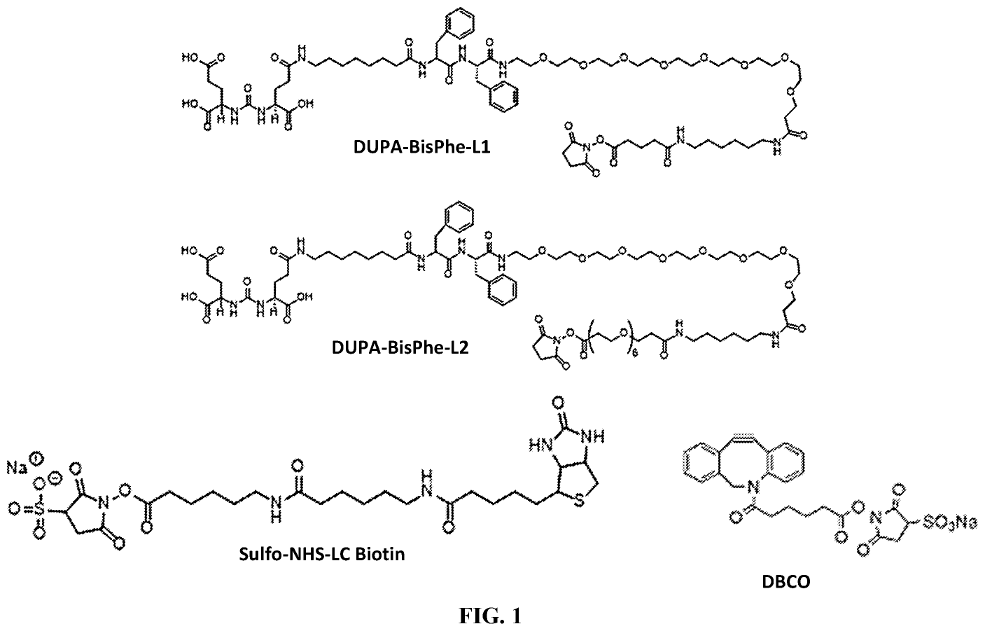

In some embodiments, a linker can include a spacer that has a length of at least 25 Angstroms, at least 50 Angstroms, at least 75 Angstroms, or at least 100 Angstroms, or, for example, can have a linker with a chain length of at least 16 atoms, at least 32 atoms, at least 50 atoms, at least 65 atoms, at least 70 atoms, or at least 75 atoms. A spacer can be of any length, but in some embodiments a spacer of a linker of a DUPA compound that connects DUPA to the functional group used for conjugation to cells can in some embodiments have a length of from about 50 angstroms to about 400 angstroms or greater, for example from about 50 angstroms to about 300 angstroms, or from about 100 Angstroms to about 400 Angstroms, from about 100 Angstroms to about 350 Angstroms, from about 50 Angstroms to about 250 Angstroms, from about 80 Angstroms to about 250 Angstroms, from about 90 Angstroms to about 250 Angstroms, from about 100 angstroms to about 250 Angstroms, from about 70 Angstroms to about 200 Angstroms, from about 80 Angstroms to about 150 Angstroms, or from about 100 Angstroms to about 150 Angstroms. Examples of linkers are shown attached to DUPA (DUPA-Bis-Phe-L1 and DUPA-Bis-Phe-L2) in FIG. 1.

While the disclosure provides efficient methods of one-step conjugation of DUPA to innate immune cells, the methods and compositions provided herein are not limited to the exemplified methods of attaching DUPA to a cell surface and resulting cell-conjugates. The inventors also contemplate that a cell, such as an NK cell, gdT cell, or macrophage can have DUPA conjugated to the cell surface by any feasible means. For example, an NK cell, macrophage, or gdT cell can have an alkyne-containing linker conjugated to the cell surface and DUPA may be attached to a linker that includes an azide (or vice versa), where the cell and antibody can be conjugated via a copper-free click reaction between the alkyne and azide.

Also provided is a population of NK cells, gdT cells, or macrophages in which cells of the population have conjugation groups or linking moieties covalently bound to the cell surface. The NK cells, gdT cells, or macrophages can be human NK cells and can be primary cells derived from a single donor or multiple donors. Alternatively the NK cells, gdT cells, or macrophages may be from a cell line. The cells can be provided in buffers or cell media and can be provided as frozen formulations, and may be, for example, pharmaceutical formulations.

Further provided are NK cell, gdT cell, or macrophage populations that have DUPA conjugated to the cell surface via a linker that comprises a functional group for cell-surface conjugation, such as, for example, an amine reactive group such as NHS. The NK cells, gdT cells, or macrophages can be human cells and can be derived from a single donor or multiple donors. In an alternative the cells can be derived from a cell line. The cells can be provided in buffers or cell media and can be provided as frozen formulations, and may be, for example, pharmaceutical formulations. Pharmaceutical formulations comprising cells can be for intravenous administration or for injection, or a pharmaceutical formulation can be a formulation in which the conjugated NK cells, gdT cells, or macrophages are provided with a matrix, gel, fiber, structure, or polymer.

Cells conjugated with DUPA can be assessed for cytotoxicity toward PSMA-expressing tumor cells using any of various cytotoxicity assays. Examples of cytotoxicity assays are dye exclusion assays, for example using the dyes trypan blue or propidium iodide; assays that detect the reduction of tetrazolium dyes such as MTT, MTS, XTT, or WST; and assays that measure leakage of lactate dehydrogenase (LDH) from non-intact cells or assay proteases (Adan et al. (2016) Curr Pharm Biotechnol. 17:1213-1221). Other viability assays detect ATP (Nowak et al. (2018) Clin Hemorheol Microcirc 69:327-336) or use labeled Annexin V to detect phosphatidylserine (PS) on the outer membrane of cells undergoing apoptosis (e.g., Goldberg et al. (1999) J. Immunol. Methods 224:1-9). Cytotoxicity toward adherent cells can also be measured as changes in electric impedance measurements when the culture vessel includes electrodes over which the cells grow. In these assays measurements are made periodically, for example, every fifteen minutes, every thirty minutes, or every hour, to assess the degree of cell death over time. See, for example, Cerignoli et al. (2018) PLoS ONE 13(3): e0193498).

The DUPA-conjugated cells in various embodiments provided herein are not genetically modified, i.e., are not modified by molecular genetic techniques including the introduction of nucleic acid constructs, nucleic acids that target expression of endogenous genes, or enzymes that modify the genome. In other embodiments the conjugated cells may have one or more introduced genetic modifications.

### DUPA-Conjugated NK Cells

Natural Killer (NK) cells are lymphocytes of the innate immunity system that are able to kill cancer cells without prior sensitization. The cytolytic behavior of NK cells is regulated by multiple receptor-mediated signals that individually may inhibit or promote cytolytic behavior. The inventors have found that conjugation of the small molecule DUPA to the surface of NK cells using a simple conjugation method results in efficient targeting and killing of prostate cancer cells by the conjugated NK cells.

An NK cell having DUPA conjugated to the cell surface can be a primary cell or a cell of a cell line. Primary cells can be cells isolated from peripheral blood or PBMCs of one or more individuals, or primary NK cells can be derived from placental tissue or umbilical cord blood. Isolation can include positively or negatively selecting NK cells from a sample, for example using antibodies bound to a solid support. The primary NK cells may be enriched following isolation from the donor source. As used herein, “enriched” can mean culturing the cells under conditions that promotes the growth of a particular cell type or subtype while not promoting the growth of another cell type or subtype that may be present in the culture. Methods of isolating and/or enriching Natural Killer cells are known in the art and some are described for example in Spanholtz et al. (2010) PLoS ONE 6 (2):e9221 (1-13); Kaur et al. (2017) Front Immunol. 8:297; Fujisaki et al. (2009) Cancer Res. 69:4010-4017; Leivas et al. (2016) Oncolmmunology 5: e1250051; U.S. Pat. Nos. 9,834,753; 9,193,953; and 9,109,202; US 2017/0029777; US 2015/0225697; US 2014/0080148; US 2013/0059379; US 2013/0011376; and WO 2020/014029, all of which are incorporated herein by reference in their entireties. Typically, one or more cytokines is included in the culture medium to promote the selective growth or survival of NK cells in culture. Such cytokines can include, without limitation, one or more of any of the following: thrombopoeitin, Flt-3L, SCF, G-CSF, GM-CSF, IL-2, IL-3, IL-6, IL-7, IL-13, IL-15, IL-17, and H9. For example, isolated NK cells Can be placed in an expansion/activation reaction mixture with any one or any combination of, for example, 2-3 cytokines, including IL-2, 1L12, IL21, IL15 and/or IL18, under conditions that are suitable for expanding and activating the isolated NK cells. In one embodiment, the expansion/activation reaction mixture also include any one or any combination of the following agents: anti-NKp46 antibody, B7-H6-Fc, anti-NKp30 antibody and/or 4-1BBL-Fc.

For example, NK cells can be directly isolated or enriched from PBMCs using density gradient centrifugation. For example, NK cells can be directly isolated/enriched from PBMCs using positive magnetic enrichment for CD56+ NK cells (e.g., using MACS separation technology including Whole Blood CD56 MicroBead Kit or Buffy Coat CD56 MicroBead Kit, both from Miltenyi BioTec). For example, a magnetic depletion step can be employed to remove CD3+T cells from PBMCs. In one embodiment, the magnetic depletion step can be employed using the MACSxpress NK Cell Isolation Kit (Miltenyi BioTec). In some embodiments, the PBMCs can be obtained from one or more healthy human donors via leukapheresis. The depleted cells can be stimulated and expanded with irradiated autologous PBMCs in the presence of OKT3 and IL-2, for example for approximately 14 days, to generate a population of NK cells that are CD3-CD16+ CD56+.

Placental-derived NK cells can be isolated from umbilical cord blood or placental perfusate, or NK cells can be differentiated from CD34⁺ hematopoietic stem cells recovered from umbilical cord blood or placental perfusate. For example, human placenta-derived NK cells can be prepared by: culturing, hematopoietic stem or progenitor cells in a, first medium comprising a stein cell mobilizing agent and thrombopoietin (Trio), followed by culturing the cells in a medium comprising a stem cell mobilizing agent and interleukin-15 (IL-15), and then culturing the cells in a third medium comprising IL-2 and IL-15 to produce a third population of cells. Human placenta-derived NK cells are prepared as described in U.S. published application No. 2019/0093081, entitled “Placental-Derived intermediate Natural Killer (PINK) Cells for Treatment of Glioblastoma”, incorporated herein by reference.

NK cell lines can be, without limitation, KYHG cells, NK92 cells, YTS cells, NK3.3 cells, NK-YS, NK-YT, NKL, NKG, MOTN-1, HANK-1, SNK-6, IMC-1, NKL cells, or other NK cell lines. An NK cell line used in the methods or compositions of the present invention can be developed for the purpose of cell therapy as set forth herein. Preferably cells of a cell line that are used in cancer therapy (i.e., a cell line having DUPA conjugated to the cell surface) is irradiated prior to delivery to a patient, where irradiation is performed at a level that allows for viability of the cells but prevents the cells from dividing. In some embodiments, the DUPA conjugated NK cells is a KHYG or KHYG-1 NK cell. The KHYG-1 cell line mediates cytolysis by granzyme M (but not granzymes A and B) together with perforin (Suck G et al., Exp Hematol 2005). KHYG-1 cells can be cultured (e.g., in RPMI 1640 medium containing 2 mM L-Glutamine, 20% FBS, 2 mM sodium pyruvate, supplemented with 450 U/ml rhlL-2) and irradiated, for example, at 10 Gy (Suck G et al. (2006) Int J Radiat Biol). Following irradiation, the cells are allowed to recover in culture for example, for twenty-four hours, and can then be frozen or used directly.

DUPA-Conjugated Gamma Delta T (gdT) Cells

Gamma delta T cells used for conjugation of DUPA to the cell surface can be isolated from blood samples, PBMCs, cord blood, or placental tissue. Methods for selecting and expanding gdT cells are known in the art and can be found in Wilhelm et al. (2014) J. Transl. Med. 12:45 as well as US 2017/0196910, WO2017/072367 and WO2018/212808, WO 2020172555, WO 2021032961, and US 20210030794, all of which are incorporated herein by reference. Commercial kits for gdT cell isolation and enrichment are also available (Stemcell Technologies, Vancouver, Calif.; Miltenyi Biotec, San Diego, Calif.). Alternatively, cells of a gamma delta T cell line may be used.

For example, gd T cells can be isolated from PBMCs using a commercially available kit such as the EasySep Human Gamma/Delta T Cell Isolation Kit (StemCell Technologies). Alternatively, gd T cells can be isolated by plating PBMCs in a culture medium containing Concanavalin A (Con A), IL-2, and IL-4 for about 1 week and culturing in a cultured medium that does not contain Con A for an additional 7 days. Another isolation method comprises plating PBMCs in a culture medium containing zolendronic acid (or another aminobisphoshonate) and IL-2 for approximately 2 days, The cells can be further cultured in a medium that does not contain zolendronic acid for an additional 12 days. Magnetic (or non-magnetic) cell sorting methods can be employed. In some cases, percent purity of the isolated gd T cell population can be determined using flow cytometry.

For example, isolation can be carried out during culturing by the addition of one or more components such as aminobisphosphonate (e.g., pamidronic acid, alendronic acid, zoledronic acid, risedronic acid, ibandronic acid, incadronic acid, or a salt or hydrate thereof) that allows the gamma delta T cells to be selectively expanded in a culture. Purification during cell culture may also be achieved by the addition of synthetic antigens such as phosphostim/bromohalohydrin pyrophosphate (BrHPP), synthetic isopentenyl pyrophosphate (IPP), (E)-4-Hydroxy-3-methyl-but-2-enyl pyrophosphate (HMB-PP) or co-culture with antigen presenting cells (APCs). The addition of such components can provide a culturing environment which allows for positive selection of gamma delta T cells typically at 70% or greater by number of total cells in the purified sample after a culture period of from 5 to 15 days, for example.

Additional factors that may be used to proliferate gamma delta T cells such as IL-2, IL-15 or IL-18 may be provided in the step of culturing the blood cells. For example, IL-2, IL-15 or IL-18 or combinations thereof may be provided in the range of 50-2000 U/ml, for example, 400-1000 U/ml, to the culturing medium.

Following isolation, gd T cells may be stimulated according to any appropriate protocol. In some cases, isolated gd T cells are stimulated using Con A. Alternatively or in addition, isolated gd T cells can be stimulated with CD3, or CD3/CD28 antagonists which promote rapid replication and expansion of the cells. Further alternatively, gd T cells can be activated through direct stimulation with ligand or antibody that binds to the gd T cell receptor (TCR).

### DUPA-Conjugated Macrophages

Macrophages are mononuclear phagocytes that are widely distributed throughout the body, where they participate in innate and adaptive immune responses. Human macrophages can be isolated by flow cytometry in view of their specific expression of proteins such as CD14, CD40, Cd11b and CD64.

Macrophages used for conjugation of DUPA to the cell surface can be derived from monocytes isolated from blood samples or PBMCs. For example, culturing of monocytes for differentiation into macrophages can be done using the cytokine M-CSF or GM-CSF in the culture medium, optionally in combination with IFNγ or IL-4. Antibodies that may be useful in enriching macrophages in a cell culture include anti-CD14, anti-CD40, anti-CD11b, and anti-CD64 antibodies. Commercial kits are available for isolating macrophages from primary monocytes (e.g., Stemcell Technologies, Vancouver, Calif.). See also Elkord et al Immunology. February 2005; 114(2):204-212); Repnik et al Journal of Immunological Methods Vol. 278, Issues 1-2, July 2003, pages 283-292); and Zhang et al (Curr. Protoc. Immunol. 83:14,1.1-14.1.14, 2008), Alternatively, macrophages may be isolated from tissue samples or may be cells of a macrophage cell line, such as U937 (Vogel et al. (2005) Environ Health Persp. 113:1536-1541), THP-1, or m2.

### Conjugation Methods

Further included herein are methods for producing DUPA-conjugated cells, such as NK cells, gdT cells, or macrophages. The methods include covalently attaching a DUPA compound that includes a DUPA moiety and a linker that comprises a functional group to NK cells, gdT cells, or macrophages. The functional group can be a functional group that reacts with thiols or amines. For example, functional groups that can be used for reaction with cell-surface sulfhydryls include, without limitation, maleimide, pyridyldithio, bromoacetyl, iodoacetyl, bromobenzyl, iodobenzyl, and 4-(cyanoethynyl)benzoyl. Functional groups used for conjugation to cell surface lysines include, as nonlimiting examples, N-hydroxysuccinimide (NHS), pentafluorophenyl, tetrafluorophenyl, tetrafluorobenzenesulfonate, nitrophenyl, isocyanate, isothiocyanate, and sulfonylchloride.

In exemplary embodiments, the DUPA compound that is conjugated to the cell surface includes an NHS functional group for conjugation to the cell surface. The linker includes a spacer that links the DUPA moiety to the NHS functional group.

The methods can include contacting the DUPA compound that includes a functional group with a population of NK cells, gdT cells, or macrophages under conditions that allow chemical conjugation of the functional group to the surfaces of the NK cells. The cells can be in an isotonic medium that may be buffered such as PBS. Reaction conditions such as concentration of reagents and reaction time and temperature can be determined empirically, but as general guidance only, the temperature can be any that allows for viability of the cells and is permissive for the conjugation reaction, for example, the conjugation reaction can be performed at temperatures ranging from about 4° C. to about 37° C., or from about 15° C. to about 37° C. In illustrative embodiments, the reaction can be performed from about 18° C. to about 32° C. Optimal concentrations of cells and DUPA compound can be determined empirically. As nonlimiting examples, the cells can be provided in the reaction at concentrations of from about 10⁵ per mL to about 10⁸ per mL, for example from about 10⁶ per mL to about 5×10⁷ per mL, and the DUPA compound can be provided at a concentration of from about 5 μM to about 1 mM, or from about 10 μM to about 800 μM, or from about 30 μM to about 600 μM. In illustrative embodiments, the DUP compound can be present in the conjugation reaction at a concentration of from about 40 μM to about 400 μM, or from about 50 μM to about 250 μM. The reaction can be incubated for minutes to hours, for example, from about 10 min to about 16 hours, and can in some exemplary embodiments be performed from about 15 min to about 2 h. The cells can be washed after the conjugation reaction from one to multiple times using a buffer such as PBS or culture medium.

### Pharmaceutical Compositions

Pharmaceutical compositions of the present invention may comprise a DUPA-conjugated cell, or a population of DUPA-conjugated cells, as described herein, in combination with one or more pharmaceutically or physiologically acceptable carriers, diluents or excipients. Such compositions may comprise buffers such as neutral buffered saline, phosphate buffered saline (PBS) and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in some embodiments formulated for intravenous administration.

For example, the pharmaceutical composition may be formulated for parenteral, systemic, intracavitary, intravenous, intra-arterial or intratumoral routes of administration which may include injection or delivery by catheter. Suitable formulations may comprise the cells in a sterile or isotonic medium. Medicaments and pharmaceutical compositions may be formulated in fluid form suitable for injection, e.g. as a liquid, solution, suspension, or emulsion, or may be formulated as a depot or reservoir, e.g. suitable for implantation in the subject's body, from which the rate of release of the cells may be controlled. Depot formulations may include gels, pastes, boluses or capsules. The preparation may be provided in a suitable container or packaging. Fluid formulations may he formulated for administration by injection or via catheter to a selected region of the human or animal body.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation. Suitable carriers, adjuvants, excipients, etc. can be found in standard pharmaceutical texts.

### Methods of Treating Cancer

Further provided is a method of treating cancer, comprising delivering a population of NK cells, gdT cells, or macrophages having DUPA covalently attached to the cell surface via a linker to a subject with cancer. In some embodiments, the method includes delivering a population of NK cells, gdT cells, or macrophages having DUPA covalently attached to the cell surface to a subject with cancer.

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinurn, mesentery, myometrium, nasopharynx, omentume, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors that express PSMA. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

The cancer may be a solid tumor that expresses PSMA, such as but not limited to PSMA-positive prostate cancers which may be metastatic to other organs or tissues. Other types of cancer that may express PSMA such as but not limited to colorectal cancer, gliomas, lung cancer, breast cancer, pancreatic cancer (Galina Barbosa et al. (2020) Cancer Imaging 20:23), may also be treated with the cells and methods provided herein.

Delivery can be, for example, by intravenous administration or injection. The DUPA-conjugated cells can be infused into the bloodstream or can be delivered into a body cavity. The conjugated cells can be delivered peritumorally or intratumorally, for example by injection.

The methods can be used to deliver an effective amount of DUPA conjugated cells to a patient having cancer, for example, having prostate cancer, including metastatic prostate cancer. An effective amount is an amount that provides a therapeutic benefit. The method can comprise giving a single does or multiple doses of DUPA-conjugated cells, where a dose can include, for example, from 10⁴ to 10¹² cells per kg of body weight.

Cell-based immunotherapy can include the transfer of primary NK cells, gdT cells, or macrophages isolated from one or multiple donors. Autologous cell-based immunotherapy can include transfer of primary cells isolated from the patient. Isolation of NK cells from PBMCs is disclosed, for example in Example 2, and in numerous references in the art. Cell-based immunotherapy can alternatively include the transfer of NK cells, gdT cells, or macrophages from cell lines, such as for example KHYG cells or NK92 cells where the NK cells of the cell lines have cell surface-conjugated DUPA. Cell lines used for adoptive cell immunotherapy can be irradiated prior to transfer to the patient so that the transferred cells do not proliferate in the patient.

Pharmaceutical compositions of DUPA-conjugated cells as provided herein may be administered in a manner appropriate to the disease to be treated. The quantity and frequency of administration will be determined by such factors as the condition of the subject, and the type and severity of the subject's disease, although appropriate dosages may be determined by clinical trials. The subject may be a human patient. For example “an effective amount”, “an anti-tumor effective amount”, “a tumor-inhibiting effective amount”, or “a therapeutic amount” can be determined by a physician with consideration of individual differences in age, weight, tumor size, extent of infection or metastasis, and condition of the patient (subject). In some embodiments, a pharmaceutical composition comprising the cells, e.g., gdT cells, NK cells, or macrophages described herein may be administered at a dosage of 10⁴ to 10⁹ cells/kg body weight, in some instances 10⁵ to 10⁶ cells/kg body weight, including all integer values within those ranges. In some embodiments, the cells, e.g., T cells described herein may be administered at 3×10⁴, 1×10⁶, 3×10⁶, or 1×10⁷ cells/kg, body weight. The cell compositions may also be administered multiple times at these dosages. The cells can be administered for example by using infusion techniques that are commonly known in immunotherapy.

The compositions described herein may be administered to a patient by intravenous infusion, trans-arterially, subcutaneously, intradermally, intratumorally, intranodally, intramedullary, intramuscularly, by intravenous (i.v.) injection, or intraperitoneally. The administration of the subject compositions may also be carried out by aerosol inhalation, injection, ingestion, transfusion, implantation, or transplantation. In some embodiments, the cell compositions, e.g., macrophage, gdT cell, or NK cell compositions, of the present invention may be administered to a patient by intradermal or subcutaneous injection. In some embodiments, the cell compositions e.g., DUPA conjugated macrophage, gdT cell., or NK cell compositions of the present invention may be administered by i.v. injection. The compositions of cells e.g., macrophage, gdT cell, or NK cell compositions, of the present invention are administered to a patient by intradermal or subcutaneous compositions, and may be injected directly into a tumor, lymph node, or site of infection.

In some embodiments, the subject (e.g., human subject) receives an initial administration of DUPA-conjugated cells, e.g., macrophages, gdT cells, or NK cells as provided herein, and one or more subsequent administrations of the DUPA-conjugated cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the DUPA-conjugated cells, e.g., macrophages, gdT cells, or NK cells as provided herein are administered to the subject (e.g., human) per week, e.g., 2, 3, or 4 administrations of the DUPA-conjugated cells, e.g., are administered per week. In one embodiment, the subject (e.g., human subject) receives more than one administration of the DUPA-conjugated (e.g., 2, 3 or 4 administrations per week) (also referred to herein as a cycle), followed by a week of no cells, and then one or more additional administration of the DUPA-conjugated cells, (e.g., more than one administration of the DUPA-conjugated cells per week) is administered to the subject. In another embodiment, the subject (e.g., human subject) receives more than one cycle of DUPA-conjugated cells, and the time between each cycle is less than 10, 9, 8, 7, 6, 5, 4, or 3 days. In one embodiment, the DUPA-conjugated cells are administered every other day for 3 administrations per week. In one embodiment, the DUPA-conjugated cells are administered for at least two, three, four, five, six, seven, eight or more weeks. The foregoing schedules are exemplary and not limiting to the methods provided herein.

### EXAMPLES

### Example 1

### Synthesis of DUPA-BisPhe-L1

For conjugation of DUPA to the surface of NK cells, the DUPA-BisPhe-L1 was synthesized by the following method depicted below.

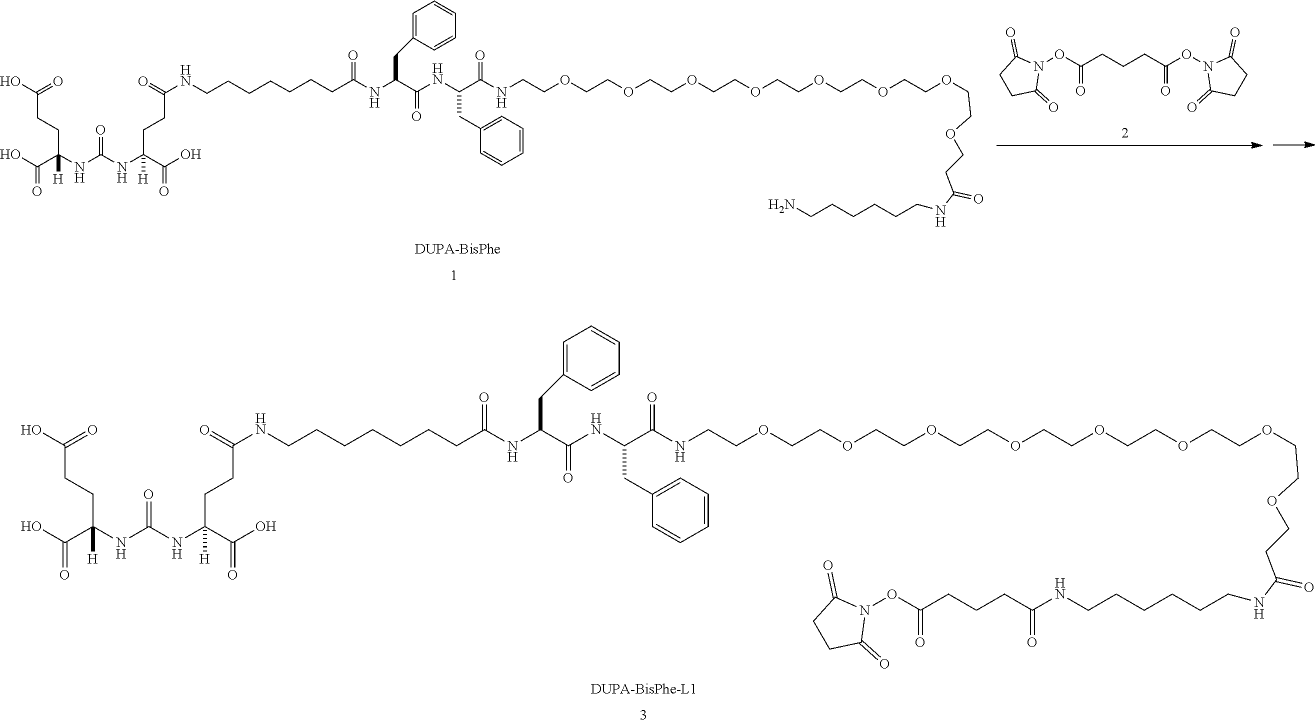 [SMILES: NCCCCCCNC(=O)CCOCCOCCOCCOCCOCCOCCOCCOCCNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O ; O=C(CCCC(=O)ON1C(=O)CCC1=O)ON1C(=O)CCC1=O ; O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCOCCOCCOCCOCCOCCOCCOCCOCCC(=O)NCCCCCCNC(=O)CCCC(=O)ON1C(=O)CCC1=O)C(=O)O)C(=O)O]

Starting material DUPA-BisPhe (1) was synthesized as described in Bioorganic & Medicinal Chemistry Letters (2017), 27(24), 5490-5495.

To a solution of compound 1 (20 mg, 15.7 μmol) in 2 mL of dimethyl formamide (DMF) was added activated ester 2 (26 mg, 79.8 μmop and 6 μL of N,N-Diisopropylethylamine (DIEA). The reaction mixture was stirred for 30 min until all of compound 1 was consumed. Then the mixture was purified by reverse phase (RP) HPLC (0.1% trifluoroacetic acid (TFA) in water/acetonitrile). The fractions containing the product were lyophilized to give DUPA-BisPhe-L1 (3) (13.3 mg) as a glassy solid.

### Example 2

### Conjugation of DUPA to NK Cells

KHYG cells (Yagita et al. (2000) Leukemia 14:922-930; Suck et al. (2005) Exp. Hematol. 33:1160-71) are CD3− cells of a Natural Killer leukemia cell line having a p53 point mutation. KHYG cells were cultured in RPMI 1640 medium including 10% FBS. Two conjugation conditions were tested, one in which the concentration of DUPA-BisPhe-L1 in the KHYG cell conjugation reaction was 200 μM, and one in which the concentration of DUPA-BisPhe-L1in the cell conjugation reaction was 353 μM.

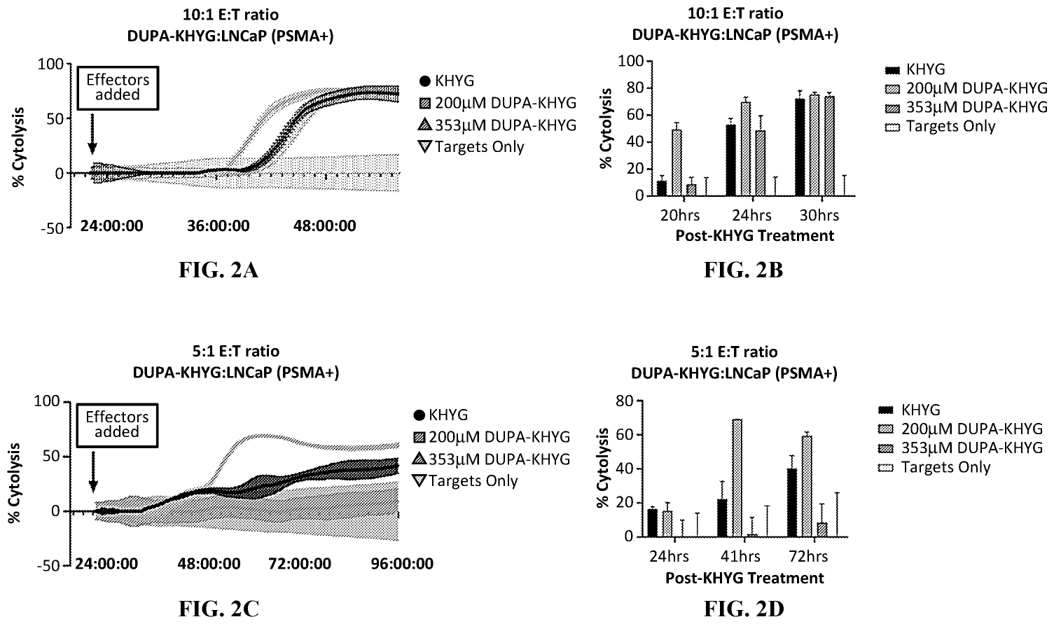

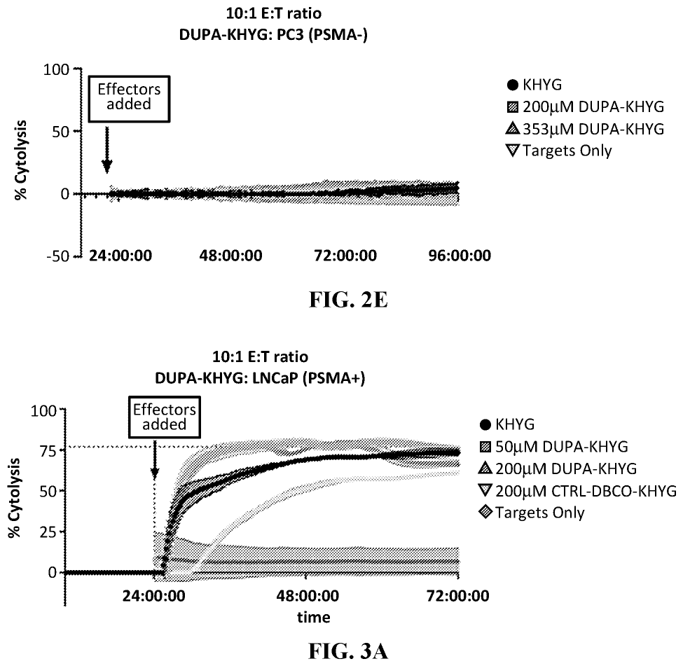

To conjugate DUPA to the surface of KHYG cells, the DUPA-BisPhe-L1 having the NHS functional group (FIG. 2A) was covalently attached to the cells via exposed lysine residues on the cell surface as follows.

A stock of 35 mM DUPA-BisPhe-L1 (FIG. 2A) in DMSO was further dissolved in PBS to a final concentration of 200 μM for use in the first set of reactions and to a final concentration of 353 μM for use in the second set of reactions. The KHYG NK cells were first extensively washed with PBS to remove components in the cell media that might interfere with the conjugation reaction. Aliquots of 5×10⁶ cells were added to the wells of a U-bottom 96-well plate, the plate was centrifuged to pellet the cells, and excess supernatant was removed. After the final wash, the pelleted cells were resuspended in either 200 μL per well of 200 μM DUPA-BisPhe-L1 or 353 μM DUPA-BisPhe-L1. Control wells were also included in which the same number of cells (5×10⁶) were resuspended in PBS that did not include the DUPA-BisPhe-L1 compound.

The plate was incubated for 30 min at room temperature with gentle shaking (60 rpm). After the reaction, the cells were washed twice with 200 μL PBS and once with 200 μL cell growth media (cells were spun down each time at 1500 rpm) to remove the excess unreacted DUPA-BisPhe-L1. After the washing step, the DUPA KHYG cell conjugates were resuspended in cell growth media awaiting in vitro analysis.

### Example 3

### Cytotoxicity of DUPA-conjugated KHYG Cells

Cytotoxicity assays were performed following the manufacturer's protocols for the XCELLIGENCE® Real Time Cell Analyzer (Acea Biosciences, San Diego, Calif.) that allows for real-time monitoring of NK cell-mediated cytolysis of target cells. In these assays, LNCaP cells (of a human PSMA-positive prostate carcinoma cell line) were used as target cells to test the cytotoxicity of DUPA-conjugated KHYG NK cells as effectors.

LNCaP cells for use as target cells in the assay were harvested in growth phase, counted, washed, and resuspended to a cell density of 3×10⁵ per mL. The target cells (100 μL) were added to the wells of the XCELLIGENCE® 96-well plate and incubated at 37° C. Cell growth was monitored as impedance values by the XCELLIGENCE® analyzer until the cells reached growth phase with a cell index value greater than 0.5, approximately 24 hours after plating.

Effector cells (KHYG cells with DUPA conjugated to the cell surface) were then added to the wells containing target cells. Target cells were either PSMA-positive LNCaP cells or PSMA-negative PC3 cells used as controls. Natural Killer KHYG cells that had the DUPA compound conjugated to the cell surface using the methods described in Example 2 were washed in culture medium (RPMI 1640 containing 10% FBS) and adjusted to a density of 6×10⁶ cells per mL. As additional controls, KHYG NK cells that had not been DUPA-conjugated were used as effector cells in additional wells. Assays were performed in duplicate.

Effector cells were added in 50 μL volumes at cell number ratios of 10:1 and 5:1 with respect to the target cells. Following addition of effector cells, the 96 well plate continued to be incubated at 37° C. and impedance measurements were taken every 15 min.

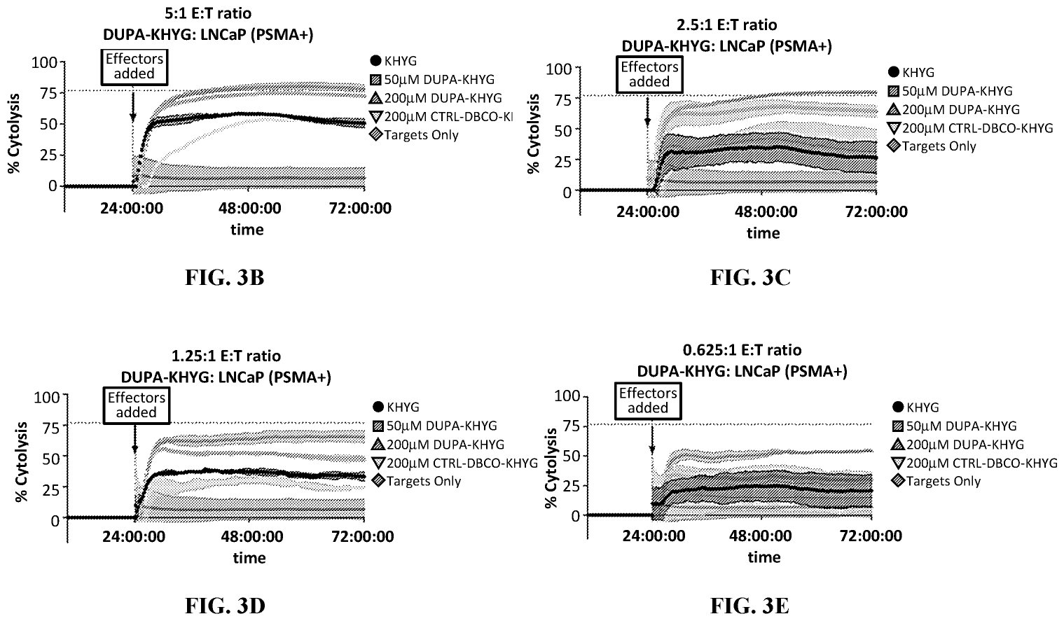

FIG. 3A shows that in assays in which the target cells were PSMA-expressing LNCaP cells and the effector cells were DUPA-conjugated KHYG cells, and the effector to target ratio was 10:1, cell killing was much more rapid when the effector cells were cell surface-conjugated with DUPA at a concentration of 200 μM relative to unconjugated KHYG cells or KHYG cells conjugated with DUPA at a concentration of 353 μM. This is demonstrated clearly in FIG. 3B, a bar chart that provides the per cent cytolysis at specific times after addition of the effector cells. Here it can be seen that DUPA-conjugated KHYG cells (conjugated in a reaction that included 200 μM DUPA) kill almost half of the target cells 20 hours after being added to the culture, whereas unconjugated KHYG cells and KHYG cells conjugated with DUPA (provided in the conjugation reaction at 353 μM) have killed 10% or fewer of the effectors by that time.

The results are even more striking in FIG. 3C, showing the results of assays conducted at an effector : target ratio of 5:1. In this case, KHYG cells conjugated with 200 μM DUPA in the conjugation reaction were far more efficient at killing target cells when compared with either unconjugated KHYG cells or KHYG cells conjugated with 353 μM DUPA in the conjugation reaction. FIG. 3D provides the percent cytolysis at 24, 41, and 72 hours after adding the effectors. At 41 hours after the 200 μM DUPA-conjugated KHYG cells were added to the effector cultures, greater than 60% cytolysis, on average, was detected in the wells, whereas an average of less than 25% cytolysis was detected in the assay wells when unconjugated KHYG cells were the effectors. Interestingly, when the effector to target ratio was 5:1, very little cytolysis was detected using KHYG cells conjugated with 353 μM DUPA in the conjugation reaction as the effector cells, even at 72 hours after the addition of the effectors to the target cells (less than 10%).

FIG. 3E provides the results of the control assays in which the target cells were PSMA negative PC3 cells. In this case, essentially no cytolysis was observed, regardless of whether DUPA was conjugated to the surface of the effector cells, and regardless of whether the cell conjugations included 200 μM or 353 μM DUPA in the conjugation reaction.

### Example 4

### Cytotoxicity of DUPA-Conjugated KHYG Cells at Low Effector:Target Ratios

Further cytotoxicity assays were performed to confirm and extend the results of Example 3. These assays also used LNCaP cells (human PSMA-positive prostate carcinoma cell line) as target cells and DUPA-conjugated KHYG NK cells as effectors, where the KHYG NK cells were conjugated under reaction conditions that included either 200 μM or 50 μM DUPA. In an additional control, the conjugation moiety dibenzocyclooctyne (DBCO), which does not specifically bind PSMA, was conjugated to KHYG cells (with a concentration of 200 μM DBCO in the conjugation reaction).

Conjugation of DUPA to KHYG NK cells was performed as described in Example 2, except that the two concentrations of DUPA-BisPhe-L1 in the conjugation reaction were 200 μM and 50 μM.

For conjugation of DBCO to KHYG cells, cells were first extensively washed with PBS to remove components in the cell media that might interfere with the first reaction step. Aliquots of 5×10⁶ cells were added to the wells of a U-bottom 96-well plate, the plate was centrifuged to pellet the cells, and excess supernatant was removed. Then, 200 μL of 200 μM of DBCO-Sulfo-NHS (FIG. 2C) dissolved in a solution of 0.56% DMSO in PBS was added to the 5×10⁶ cells in the wells of the U-bottom 96-well plate. The plate was incubated for 30 min at room temperature with gentle shaking (60 rpm). After the reaction, the cells were washed twice with 200 μL PBS and once with 200 μL cell growth media (cells were spun down each time at 1500 rpm) to remove the excess unreacted DBCO-Sulfo-NHS.

The cytotoxicity assays were performed as described in Example 3, except that the number of target cells per well was 2.5×10⁴ (in 100 μl growth medium) and effector cells were added at ratios of 10:1, 5:1, 2.5:1, 1.25:1, and 0.625:1 by diluting the effectors to the appropriate cell concentration prior to adding them to the assay wells that included targets. Natural Killer KHYG cells that had the DUPA compound conjugated to the cell surface using the methods described in Example 2 were washed in culture medium (RPMI 1640 containing 10% FBS) and adjusted to a density of 2.5×10⁵ cells per mL prior to plating. For the assays, effector cells (unconjugated KHYG cells or KHYG cells with either DUPA or DBCO conjugated to the cell surface) were added in a volume of 50 μl to the wells containing LNCaP target cells, and as controls were also added to wells that included PC3 (PSMA negative) target cells. As additional controls, some target cell wells did not receive effector cells (“Targets Only” wells). Following addition of effector cells, the 96 well plate continued to be incubated at 37° C. and impedance measurements were taken every 15 min.

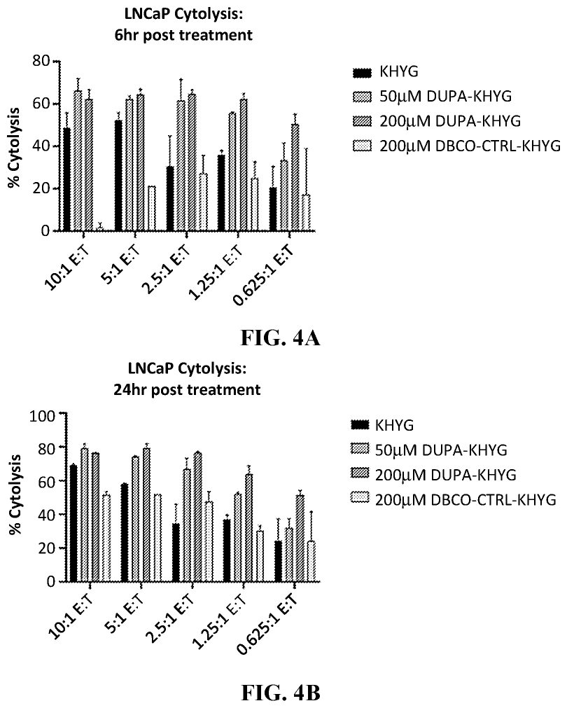

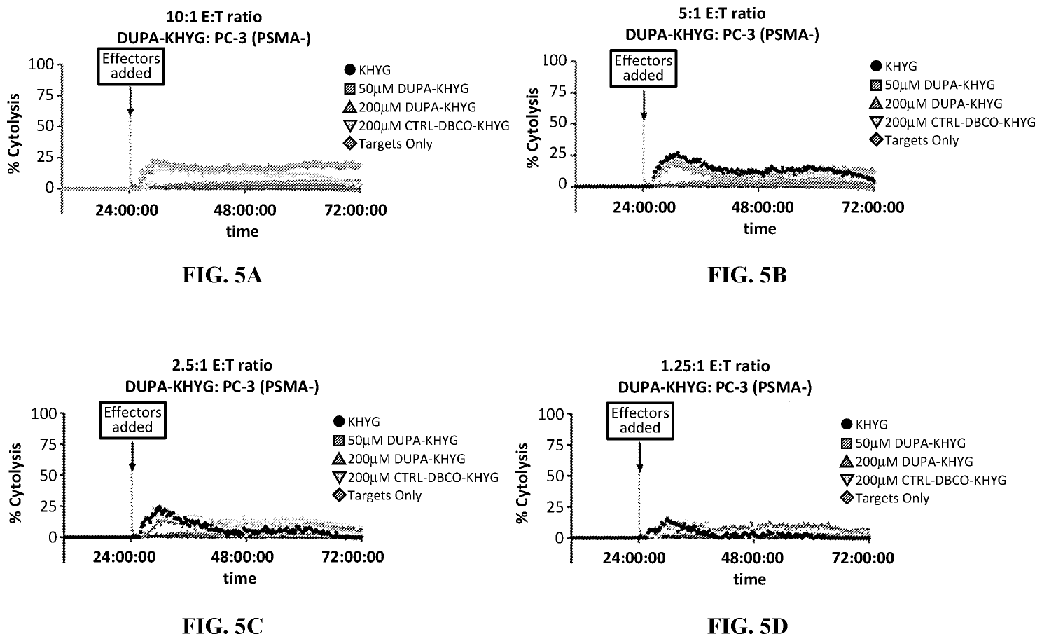

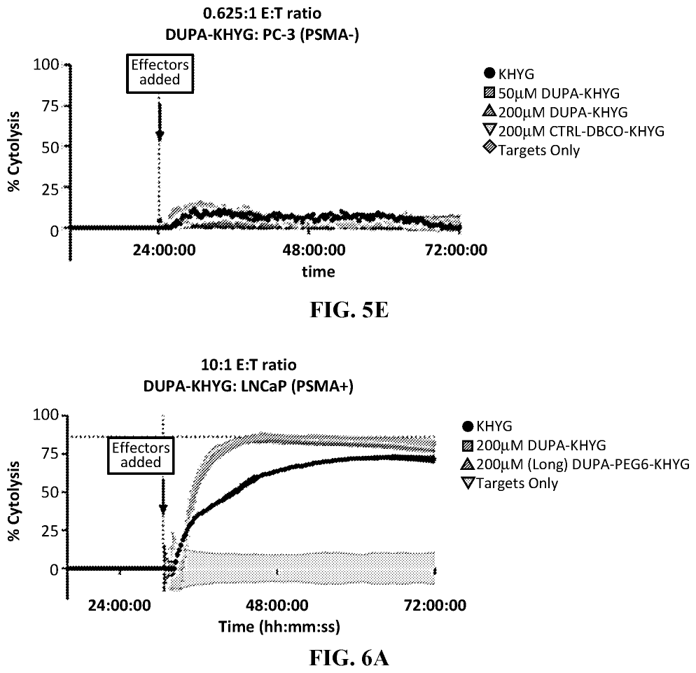

FIG. 4A shows that in assays in which the effector to target ratio was 10:1 and the target cells were PSMA-expressing LNCaP cells, KHYG cells conjugated with DUPA (at concentrations of either 50 μM or 200 μM in the conjugation reaction) were more efficient at killing target cells than unconjugated KHYG cells or DBCO-conjugated KHYG cells. Although in these assays the maximum level of cytolysis by DUPA-conjugated KHYG cells reached was similar to the maximum level of cytolysis attained by non-conjugated KHYG cells 72 hours into the assay (48 hours after the addition of effector cells), the onset of cytolysis of target cells by DUPA-conjugated KHYG cells reached the maximum level more quickly as compared with either non-conjugated KHYG cells or DBCO-conjugated KHYG cells. Cytolysis by DBCO-conjugated KHYG cells was delayed relative to that of both unconjugated KHYG cells and DUPA-conjugated KHYG cells and did not reach the level of cytolysis of DUPA-conjugated KHYG cells. These results can be clearly seen in FIG. 5A and 5B, where the 6 hour time point and 24 hour time point refer to 6 hours and 24 hours after the addition of effector cells, corresponding to 30 hours and 48 hours in the graphs of FIG. 4, demonstrate that at the 10:1 ratio DUPA-conjugated KHYG cells kill more quickly and have killed a higher percentage of cells by 24 hours after they are added to the assays with respect to both non-conjugated KHYG cells and KHYG cells with conjugated DBCO.

At a 5:1 effector:target ratio (FIG. 4B), DUPA-conjugated effectors reached higher levels of cytolysis (approximately 75%) than either unconjugated effectors or DBCO-conjugated effectors, with clearly increased effectiveness over non-conjugated KHYG cells. DUPA-conjugated KHYG cells were more effective than both non-conjugated KHYG cells and DBCO-conjugated KHYG cells at 6 and 24 hours after their addition to the assay (FIGS. 5A and 5B).

At ratios of effector to target cells of 2.5:1 and below (FIGS. 4C, 4D, and 4E, FIGS. 5A and 5B), killing was faster with DUPA-conjugated KHYG cells and the maximum percentage of cytolysis using DUPA-conjugated KHYG cells was higher than the maximum percentage cytolysis with either unconjugated KHYG cells or DBCO-conjugated KHYG cells. At these ratios, a dose response was observed with respect to the concentration of DUPA used in the conjugation reaction, with the effectors conjugated with a higher concentration of DUPA (200 μM) demonstrating higher levels of cytolysis that effectors conjugated with 50 μμM DUPA when the effector:target ratio was 2.5:1, 1.25:1, or 0.625:1 (FIGS. 4C, D, and E) as clearly seen in the bar graphs of FIGS. 5A and 5B, providing the percent cytolysis at 6 and 24 hours after the addition of target cells.

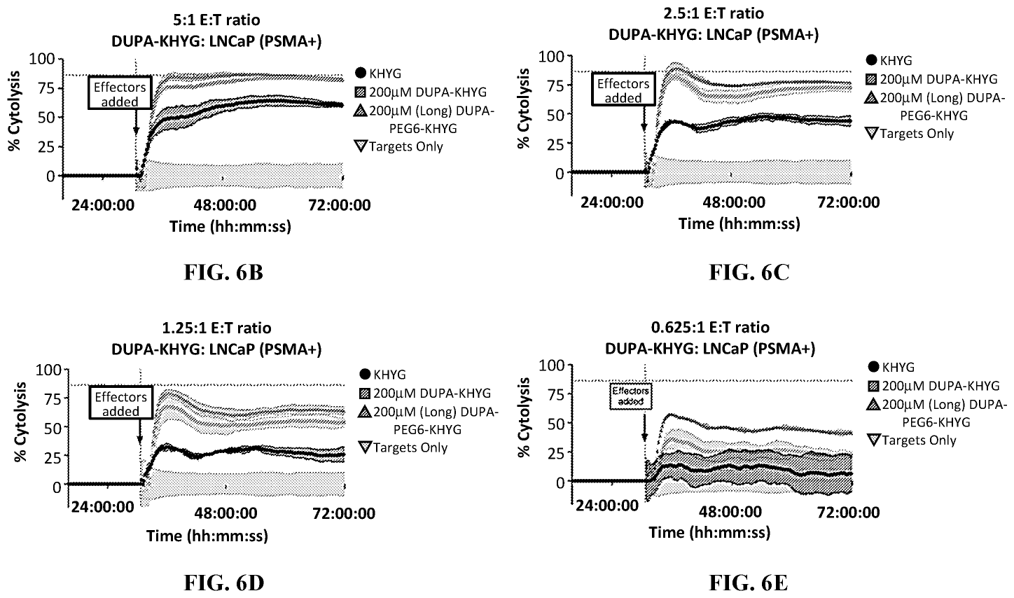

The same assays conducted with PSMA-negative PC3 cells as the target cells showed that these cells are not killed by unconjugated KHYG cells or KHYG cells conjugated with either DUPA or DBCO regardless of the effector to target cell ratio (FIGS. 6A-E). Thus conjugation of DUPA to the surface of NK cells has been shown to result in the specific targeting and cytolysis of the PSMA-positive tumor cells. DUPA-conjugated NK cells show marked increased effectiveness in cytolysis of PSMA-positive cells with respect to NK cells that do not have cell surface-conjugated DUPA, particularly at lower (less than or equal to 2.5:1) effector:target cell ratios.

### Example 5

### Synthesis of DUPA-BisPhe-L2

To determine whether compounds that included linkers of different lengths were also effective when conjugated to NK cells, DUPA-BisPhe-L2, having an additional PEG sequence proximal to the NHS group, was prepared as shown.

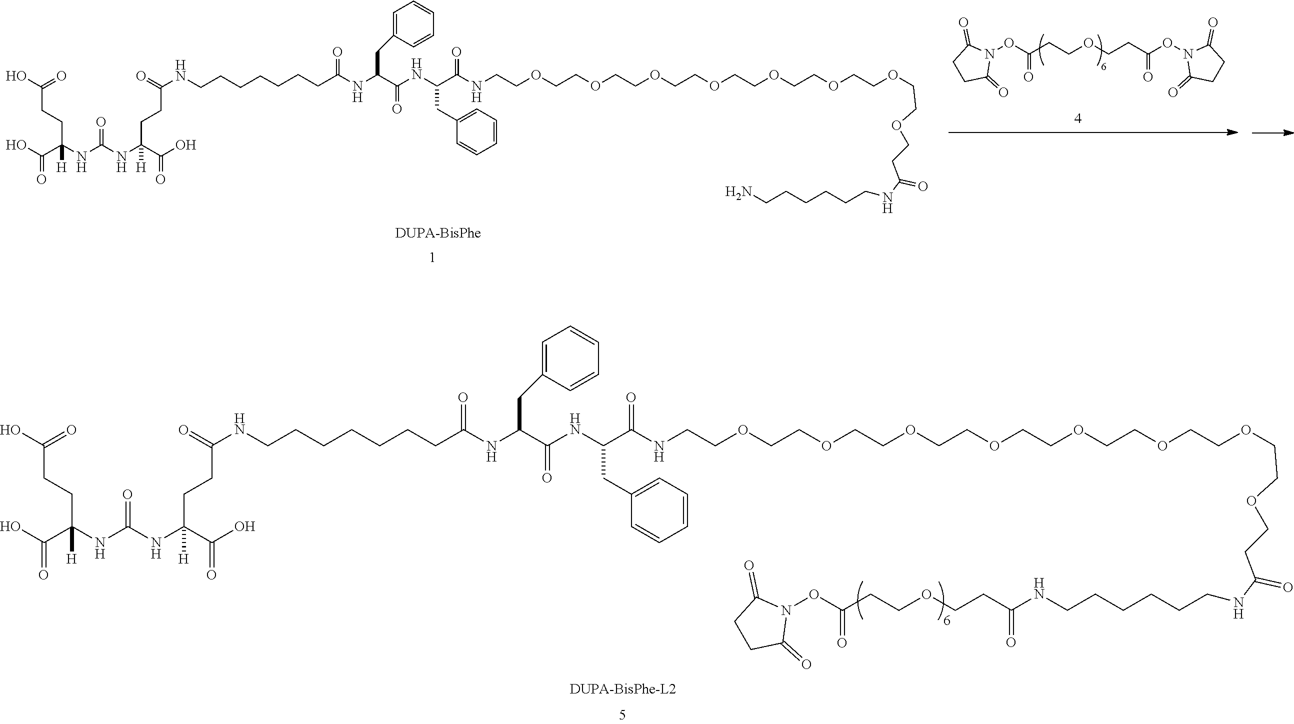

Briefly, to a solution of compound 1 (5.0 mg, 3.9 μmol) in 1.5 mL of DMF was added activated ester 4 (10 mg, 17 μmop and 3.5 μL of DIEA. The reaction mixture was stirred for 10 min until all compound 1 was consumed. Then the mixture was purified by RP HPLC (0.1% TFA in water/acetonitrile). The fractions containing the product were lyophilized to give DUPA-BisPhe-L2 (5) (5.7 mg) as a glassy solid.

### Example 6

### Cytotoxicity of DUPA-BisPhe-L2 Conjugated KHYG Cells

Cytotoxicity assays were performed on KYHG cells conjugated with either 200 μM DUPA-BisPhe-L1 linker, having a spacer length of 55 atoms or approximately 82 Angstroms (FIG. 2A) or 200 μM DUPA-BisPhe-L2 linker, having a spacer length of 72 atoms or approximately 108 Angstroms (FIG. 2B) as described in Example 2. The assays were performed using the XCELLIGENCE® Real Time Cell Analyzer (Acea Biosciences, San Diego, Calif.) as described in Example 3, except that 2×10⁴ LNCaP target cells in volume of 100 μl were plated in each well. Non-conjugated KHYG cells were used as control effectors. Additional controls were wells that included target cells only. Identical assays were also conducted using the cells of the PSMA-negative human prostate cancer cell line PC3 instead of PSMA-positive KYHG cells as targets.

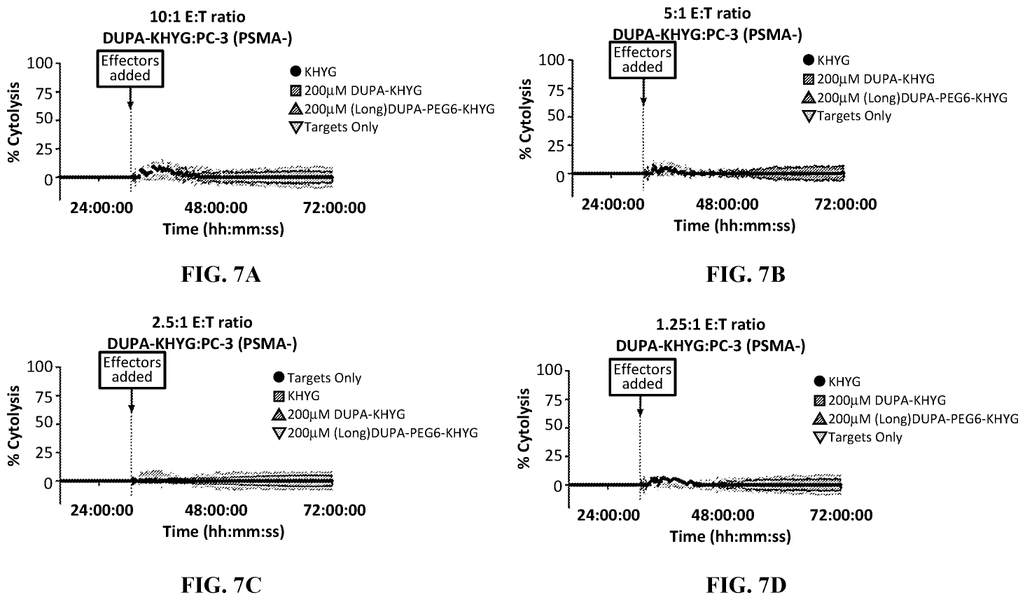

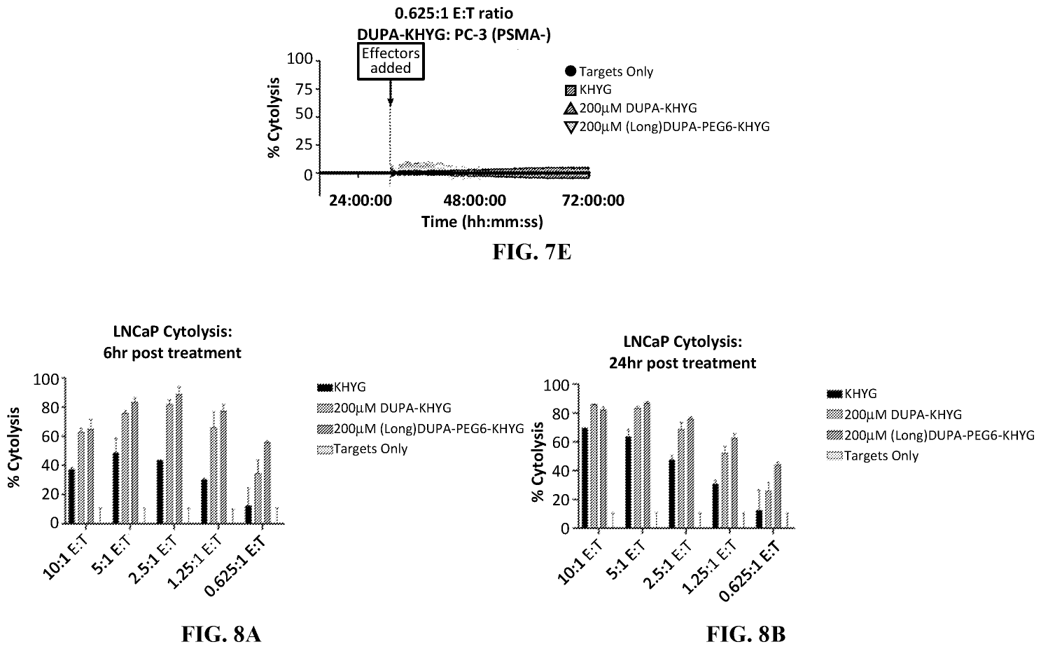

FIGS. 7A-E provide graphs of percent cytolysis over time in culture, with FIGS. 8A-E providing the corresponding controls using PSMA-negative PC3 cells as targets. FIGS. 8A-E demonstrate that none of the effectors tested (non-conjugated KHYG cells, DUPA-BisPhe-L1-conjugated KHYG cells, and DUPA-BisPhe-L2-conjugated KHYG cells) were able to kill the PSMA-negative PC3 cells.

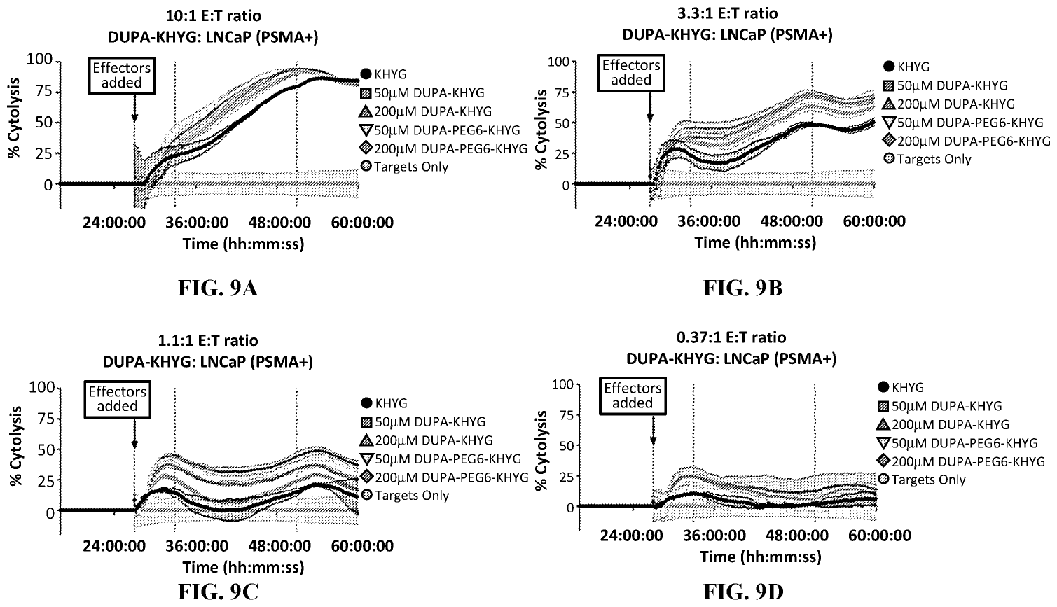

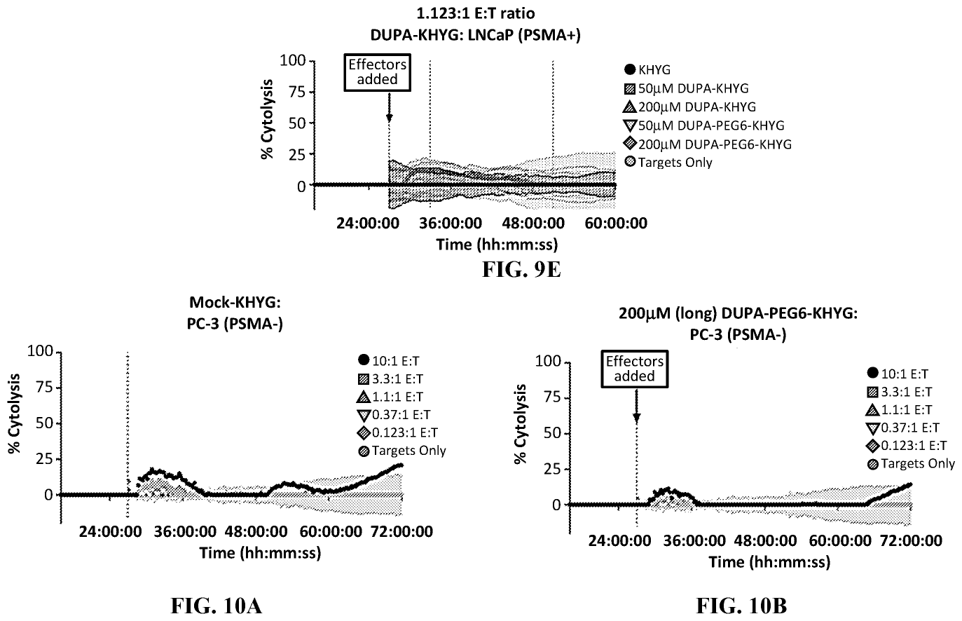

FIG. 7A shows the results of assays in which the target cells were PSMA-expressing LNCaP cells and the effector cells were DUPA-conjugated KHYG cells, where the effector to target ratio was 10:1. At this target:effector ratio, the efficiency of target cell killing was essentially identical when either the DUPA-BisPhe-L1 compound or the DUPA-BisPhe-L2 compound having a longer linker was conjugated to the effector cells. As seen in the previous study (Example 3; FIG. 4A), at the 10:1 target:effector ratio non-conjugated KHYG cells also demonstrated cytotoxicity, although the killing was much slower as compared with conjugated cells, as can be seen by the percent cytolysis observed at the 6 hour time point in FIG. 9A.

At target:effector ratios of 5:1 and 2.5:1 (FIGS. 7B and 7C) the effectors conjugated with the longer L2 linker compound achieved a slightly higher percent cytolysis at an earlier time point than the L1 linker compound-conjugated effectors, and effector cells conjugated with either the DUPA-BisPhe-L1 or DUPA-BisPhe-L2 compound were much more effective than non-conjugated KHYG cells at killing target cells throughout the assay. Higher levels of cytolysis are clearly seen at 6 hours and 24 hours after effector addition, as depicted in the bar graphs of FIG. 9A and 9B.

At the 1.25:1 target: effector ratio, KYHG cells conjugated with DUPA-BisPhe-L2 clearly killed a higher percentage of target cells than KYHG cells conjugated with DUPA-BisPhe-L1 (FIG. 7D), which can be seen at both the 6 hour and 24 hour timepoints (FIGS. 9A and 9B). At this target: effector ratio, KHYG cells conjugated with either DUPA-BisPhe-L1 or DUPA-BisPhe-L2 kill a far higher percentage of PSMA-positive target cells than are killed by non-conjugated KHYG cells.

At the lowest effector:target cell ratio, 0.625:1, non-conjugated KHYG cells are not effective against the PSMA-positive LNCaP target cells (FIG. 7E). Strikingly however, DUPA-BisPhe-L1-conjugated KYHG cells are able to kill approximately 35% of the targets by the 6 hour time point, while DUPA-BisPhe-L2-conjugated KYHG cells are able to kill at least 55% of the target cells (FIG. 9A) by this time. The cytotoxicity of DUPA conjugated KYHG cells is also seen at the 24 hour-post effector addition time point (FIG. 9B), where the cells conjugated with the DUPA compound having the longer linker (DUPA-BisPhe-L2) demonstrate the highest level of cytotoxicity.

### Example 7

### Cytotoxicity of KHYG Cells Conjugated with DUPA-BisPhe-L1 and DUPA-BisPhe-L2 Conjugated at Different Concentrations.

An additional set of assays was performed to compare the cytotoxicity of KHYG cells conjugated with different concentrations of DUPA-BisPhe-L1 and DUPA-BisPhe-L2 toward PSMA-positive target cells. KHYG cells were conjugated in reactions that included either 1) 50 μM DUPA-BisPhe-L1 linker (FIG. 2A), 2) 200 μM DUPA-BisPhe-L1 linker, 3) 50 μM DUPA-BisPhe-L2 linker (FIG. 2B), or 4) 200 μM DUPA-BisPhe-L2 linker. In these experiments, 2.5×10⁴ LNCaP cells were plated per well as targets, and the DUPA-conjugated KHYG effector cells were added to the target cells at ratios of 10:1, 3.3:1, 1.1:1, 0.37:1, and 0.12:1. Controls included assays in which the KHYG effector cells were used in mock conjugation reactions that did not include any DUPA-BisPhe linker. Additional control assays were performed using PC3 prostate cancer cells that do not express PSMA. Assays with LNCaP target cells were performed in duplicate.

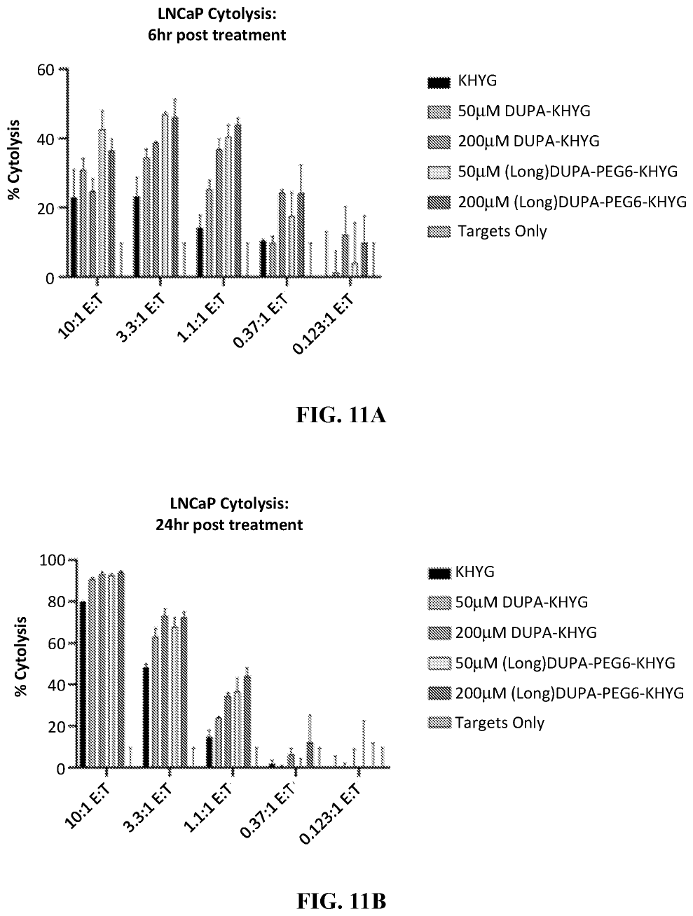

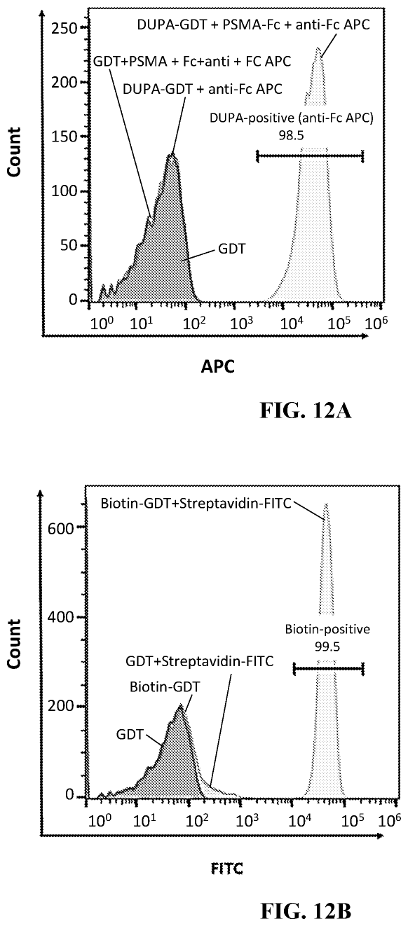

Assays were performed on the XCELLIGENCE® Real Time Cell Analyzer (Acea Biosciences, San Diego, Calif.) as described in Example 3. FIG. 10A-E provides the data from the real time impedance assays using LNCaP cells as targets, while FIG. 11A provides the results of the impedance assay using mock conjugated KHYG cells (i.e., non-conjugated KHYG cells) as effectors against PSMA- PC3 cells and FIG. 11B provides the results of the impedance assay using 200 μM DUPA-BisPhe-L2 linker-conjugated KHYG cells as effectors against PSMA− PC3 cells. At the higher target:effector ratios of 10:1 and 3.3:1, all of the DUPA-conjugated KHYG cells showed a higher level of cytotoxicity than non-conjugated KHYG cells, with somewhat faster killing exhibited by the KHYG cells conjugated with the DUPA-BisPhe-L2 linker as compared with KHYG cells conjugated with the DUPA-BisPhe-L1 linker, although the level of cytotoxicity reached at 24 hours after the addition of effectors was essentially identical (FIGS. 12A and 12B). At lower effector:target ratios of 1.1:1, 0.37:1, and 0.123:1, a clear dose response was evident for both DUPA-BisPhe-L1-conjugated cells and DUPA-BisPhe-L2-conjugated cells, where conjugation with 200 μM DUPA compound resulted in more effective NK cells than conjugation with 50 μM DUPA compound. The longer spacer of DUPA-BisPhe-L2 appears to result in slightly higher cytolysis of targets at most effector:target ratios, which can be seen most clearly in FIGS. 12A and 12B.

### Example 8

### Isolation and Expansion of Primary NK Cells

PBMC are isolated from human blood by density gradient centrifugation using Lymphoprep™ (StemCell Technologies). The isolated PBMCs are resuspended in OpTmizer™ T Cell Expansion Medium (Thermo Fisher) with 5% CTS immune cell serum replacement (Thermo Fisher)(SR) or, alternatively, 5% human AB serum (AB). The cells are cultured in coated T25 flasks (10×10⁶ cells in 10 mL/flask) in OpTmizer™ medium supplemented with cytokines. On day 4 the medium is removed and replaced with fresh 10 ml of medium plus cytokines containing either 5% SR or 5% AB. On day 7, cells are counted and evaluated for NK cell content by staining with anti-human CD3 conjugated to APC and anti-CD56 conjugated to PE, after which the cells are re-plated with fresh culture medium with cytokines in fresh coated T75 flasks. The medium is replenished again as before on day 10, and on day 14 the cells are harvested from the flasks and collected by centrifugation at 400×g for 5 minutes. The pelleted cells are resuspended, counted, and phenotyped.

The culturing enriches CD56-positive NK cells in the culture significantly by day 12, such that the NK cells may make up at least 90% or at least 95% of the culture, with a concomitant essentially complete loss of T cells (CD3-positive cells) from the culture. Such primary NK cells may be used for conjugation of DUPA to the cell surface for cell-based therapies.

### Example 9

### Isolation of gdT Cells

Gamma delta T cells (gdT cells) were isolated from peripheral blood mononuclear cells (PBMCs) using the Stemcell Technologies Human Gamma/Delta T Cell Isolation Kit (Stemcell Technologies, Seattle, Wash.). PBMCs were isolated from Leukopaks ordered through HemaCare and frozen at a concentration of 1×10⁸ cells per ml in vials. Freshly thawed PBMC suspensions were suspended in 30 mL Dulbecco's Phosphate Buffered Saline (DPBS) containing 25% fetal bovine serum (FBS). Ten vials were thawed into 30 mL DPBS medium containing 25% FBS in a single 50 mL centrifuge tube, resulting in approximately 8×10⁸-1×10⁹ cells per 50 mL tube. The PBMCs were passed through a 40 μm cell strainer and cell number was determined. Approximately 3×10⁵ cells were reserved for flow cytometry and the remainder were harvested by centrifugation. The supernatant was removed and the cell pellet was resuspended in 60 μL MACS separation buffer (Miltenyi Biotech, San Diego, Calif.) per 10⁷ cells in a 50 mL tube, to which 20 μL FcR blocking reagent per 10⁷ cells was added. The cells were incubated with blocking reagent for 5 minutes at room temperature, after which 12.5 μL of EasySep™ Human Gamma/Delta T Cell Isolation Cocktail (Stemcell Technologies, Seattle, Wash.) was added per 5×10⁷ cells. After mixing briefly, the cells were incubated a further 15 min at room temperature with mixing on a plate shaker. The cells were then washed to remove unbound primary antibody by adding 1-2 mL of buffer per 10⁷ cells and centrifuged at 1400 rpm for 5 min. The supernatant was aspirated and the cell pellet was resuspended in MACS separation buffer (80 μL per 10⁷ cells).

For pan cell depletion, magnetic particles (anti-biotin microbeads, Miltenyi Biotech) were vortexed before removing 12.5 μL of suspended beads per 5×10⁷ cells and adding to the suspended cell preparation. The cells and magnetic beads were incubated for 10 min without shaking at room temperature, and then additional MACS separation buffer was added to bring the volume up to 25 mL (if the original volume was less than 10 mL) or 50 mL (if the original volume was greater than 10 mL). The cells were pipeted up and down gently 2-3 times to mix and the tube (without lid) was placed into the magnet stand (MACS Column Separator, Miltenyi Biotec) for 10 min at RT. The enriched cell suspension was carefully pipeted into a new 50 mL tube. The cells were centrifuged at 1400 rpm for 5 min, after which the supernatant was removed. The cells were then resuspended at approximately 10⁷ cells/mL in MACS separation buffer.

2.5 μL anti-TCR α/β-biotin human antibodies (Miltenyi Biotec) were added per 10⁷ cells to the pan cell depleted cells and the antibodies and cells were mixed with pipette tips and then incubated for 10 min at 4° C. in the dark. The cells were then washed by adding 13 mL MACS separation buffer, transferring the suspended cells to a 15 mL tube, centrifuging at 1400 rpm for 5 min, and aspirating the supernatant. The wash was repeated and the final pellet was resuspended in 97.5 μK MACS separation buffer per 10⁷ cells and 2.5 ul/10⁷cells anti-biotin Microbeads were then added to the cells. The suspension was pipeted a few times to mix, mixed with pipet tips, and incubated 15 min in the dark at 4° C.

The cells were then washed by adding 13 mL buffer and centrifuging the sample at 1400 rpm for 5 min. The supernatant was aspirated completely and the cells were resuspended in up to 500 μL MACS separation buffer/10⁸cells.

For depletion of α/β T cells, the LD column (Miltenyi Biotec) was rinsed with 2 mL of MACS separation buffer and the cell suspension was applied to the column. The flow through of unlabeled cells was collected and the column was washed 5 times with 1 mL of buffer each time. The washes were added to the flow through and the cell number was determined. An aliquot of 3×10⁵ cells was removed for flow cytometry to assess cell purity. The remaining cells were spun down and resuspended in T cell medium to a concentration of 2×10⁶ cells per mL and dispensed into wells of a 6 well culture plate.

For expansion of T cells, T cell TransAct solution (Miltenyi Biotec, 5 μL per 10⁶ cells) was added to the cells in T Cell Medium, which was T cell OpTmizer™ CTS™ Medium (Fisher Scientific) modified to include 1% glutamax, 5% human serum, 26 ml of OpTmizer™ T-Cell Expansion Supplement, 1:1000 gentamicin, and 300U IL-2. The plate was placed in a cell incubator for 2-3 days. The culture medium was then exchanged with fresh T cell medium without added T cell TransAct solution (Miltenyi Biotec).

On day 9, during the exponential phase of T cell growth, the gdT cells were transferred to a suitable tissue culture bag. The cells were maintained at a density of 0.5×10⁶ cells/ml. On day 13, the cells were counted after resuspension and transferred to a tissue culture bag to keep the cell density at 0.5×10⁶ cells/ml. On day 16, the cells were again counted and thereafter the cell density was maintained at 1×10⁶ cells/ml in culture medium containing 300 U/ml rIL-2 in a tissue culture bag. On day 20, the cells were counted and frozen.

Example 10. Conjugation of DUPA to gdT Cells.

gdT cells were cultured in RPMI 1640 medium including 10% FBS. All gdT cells were reacted with 200 μM of DUPA-BisPhe-L1 (short linker), DUPA-BisPhe-L2 (long linker), or with the non-PSMA targeting small molecule compound as control, EZ-Link™ Sulfo-NHS-LC-Biotin (Thermo Fisher Scientific) (FIG. 1).

To conjugate DUPA to the surface of gdT cells, the DUPA-BisPhe-L1, DUPA-BisPhe-L2, or Sulfo-NHS-LC-Biotin having NHS functional group was covalently attached to the cells via exposed lysine residues on the cell surface as follows.

Stocks of 36-50 mM small molecule compounds (DUPA-BisPhe-L1, DUPA-BisPhe-L2, or Sulfo-NHS-LC-Biotin) in DMSO were further dissolved in PBS to final concentrations of 200 μM (0.4-0.55% final DMSO concentration in solution). The gdT cells were extensively washed with PBS to remove components in the cell media that might interfere with the conjugation reaction. Aliquots of 5×10⁶ gdT cells were added to the wells of a U-bottom 96-well plate, the plate was centrifuged to pellet the cells, and excess supernatant was removed. After the final wash, the pelleted cells were resuspended in either 200 μL per well of 200 μM DUPA-BisPhe-L1, 200 μM DUPA-BisPhe-L2, or 200 μM Sulfo-NHS-LC-Biotin. An additional control well was also included in which the same number of cells (5×10⁶) were resuspended in PBS containing 0.4-0.55% final DMSO concentration but no compound for conjugation.

The plate was incubated for 30 min at room temperature with gentle shaking (60 rpm). After the reaction, the cells were washed twice with 200 μL PBS and once with 200 μL cell growth media (cells were spun down each time at 1500 rpm) to remove the excess unreacted small molecules. After the final washing step, the DUPA-gdT cell conjugates and Biotin-gdT cell conjugates were resuspended in cell growth media awaiting in vitro analysis.

To confirm the conjugation of DUPA to the cell surface of gdT cells, gdT cells that had been reacted with DUPA-BisPhe-L1 and DUPA-BisPhe-L2 were analyzed by flow cytometry. Aliquots of 1×10⁶ unconjugated and DUPA-conjugated gdT cells were labeled with or without 40 μL of 25 μg/mL PSMA-Fc in 90 μL PBS by incubation at room temperature for 30 min in individual microcentrifuge tubes. Cells not treated with PSMA-Fc (resuspended in PBS only) served as controls. After incubation, the cells were washed with 400 μL PBS and spun down at 150×g for 3 min. After removal of PBS, cells were resuspended in the presence or absence of 90 μL 200 μg/mL APC-anti-human IgG Fc antibody for detection. Cells not labeled with APC-anti-human IgG Fc (resuspended in PBS without APC-anti-human Fc) were included as further controls. The cells were incubated for 30 min at room temperature, washed with 400 μL PBS, spun down at 150×g for 3 min, and resuspended in PBS. FIG. 12A shows the confirmation of the covalent cell surface modification of gdT cells with DUPA small molecule compounds via flow cytometry. FIG. 12A shows that only the cells that were conjugated with DUPA and then incubated with PSMA, followed by labeling with APC-anti-human IgG Fc, were labeled, demonstrating the presence of DUPA on the cell surface of approximately 98.5% of these cells. Cells not conjugated to DUPA (or conjugated to DUPA but not incubated with PSMA-Fc) were not labeled with APC.

For gdT-biotin reaction confirmation, aliquots of 1×10⁶ unconjugated and biotin-conjugated gdT cells were labeled with 1 μL of 500 μg/mL Streptavidin-FITC in 90 μL PBS by incubation at room temperature for 30 min in individual microcentrifuge tubes. Cells incubated in PBS in the absence of Streptavidin-FITC served as controls. Cells were incubated for 30 min at room temperature, washed with 400 μL PBS, and spun down at 150×g for 3 min. Finally, all cells, including controls receiving no treatment, were resuspended in PBS and analyzed by flow cytometry. FIG. 12B provides the results of flow cytometry analysis with control cells conjugated with biotin attached to an NHS functional group for binding the cells surface, where detection was by binding of streptavidin-FITC. The results demonstrate that approximately 99.5% of the reacted cells did have biotin bound to the surface.

### Example 11

### Cytotoxicity of DUPA-Conjugated gdT Cells

Cytotoxicity assays were performed following the manufacturer's protocols for the XCELLIGENCE® Real Time Cell Analyzer (Acea Biosciences, San Diego, Calif.) that allows for real-time monitoring of gdT cell-mediated cytolysis of target cells. In these assays, LNCaP cells (of a human PSMA-positive prostate carcinoma cell line) were used as target cells to test the cytotoxicity of DUPA-conjugated gdT cells as effectors.

LNCaP cells for use as target cells in the assay were harvested in growth phase, counted, washed, and resuspended to a cell density of 5×10⁵ cells per mL. The target cells (50 μL) were added to the wells of the XCELLIGENCE® 96-well plate and incubated at 37° C. PC-3 cells (a human PSMA-negative prostate carcinoma cell line) were used as a negative control cell line in the assay, resuspended at a cell density of 2×10⁵ cells per mL. Cell growth was monitored as impedance values by the XCELLIGENCE® analyzer until the cells reached growth phase with a cell index value greater than 0.5, approximately 24 hours after plating.

Effector cells (gdT cells with DUPA conjugated to the cell surface, gdT-DUPA-L1 and gdT-DUPA-L2) were then added to the wells containing target cells. Target cells were either PSMA-positive LNCaP cells or PSMA-negative PC3 cells used as control. gdT cells that had the DUPA compound conjugated to the cell surface using the methods described in Example 10 were washed in culture medium (RPMI 1640 containing 10% FBS) and adjusted to a density of 5×10⁶ cells per mL. As controls, gdT cells that had not been conjugated to either DUPA or biotin (gdT) as well as gdT cells that had been conjugated with the non-PSMA targeting compound (gdT-biotin) were used as effector cells in additional wells. Assays were performed in duplicate.

Effector cells were added in 50 μL volumes at cell number ratios starting at 3:1 with respect to the target cells. A three-fold serial dilution was made in cell growth media for a total of four Effector:Target ratio treatments, namely 3:1, 1:1, 0.3:1, and 0.1:1. Following addition of effector cells, the 96-well plate continued to be incubated at 37° C. for at least 72 h and impedance measurements were taken every 15 min.

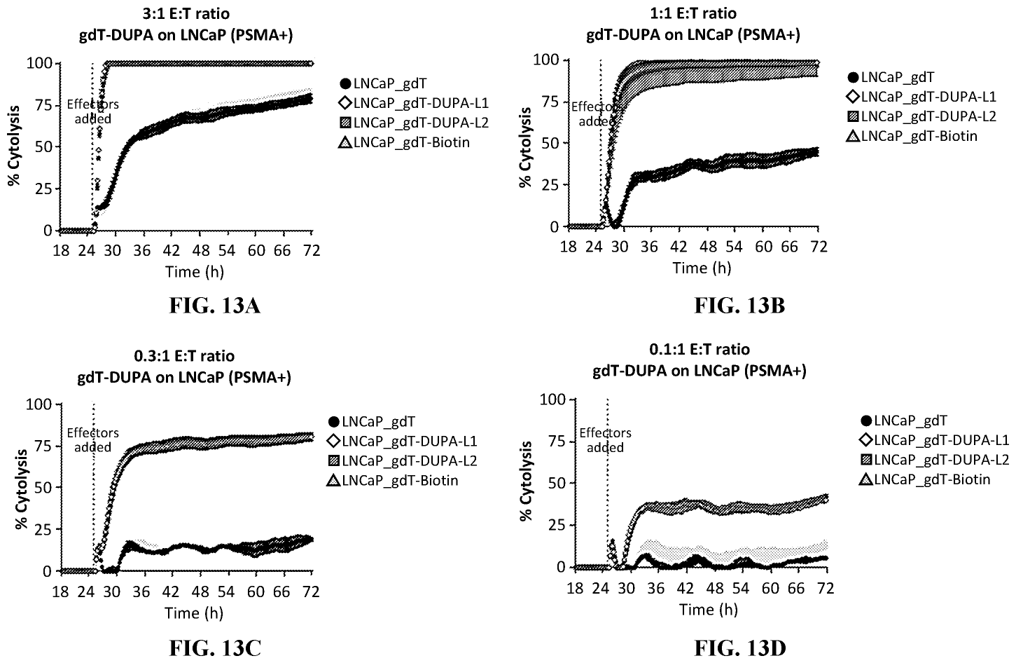

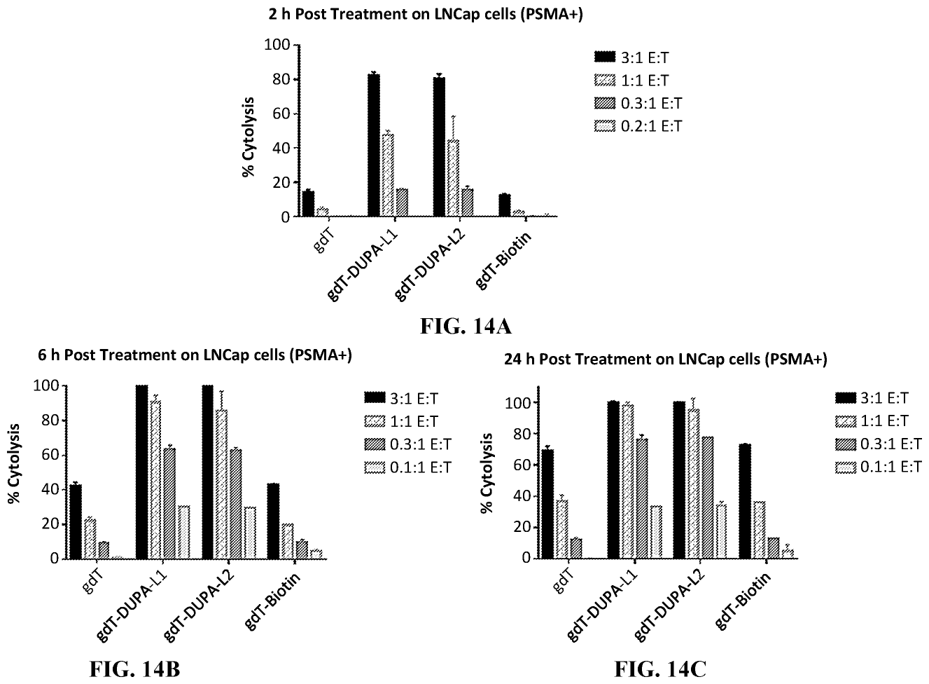

FIGS. 13A-D show that in assays in which the target cells were PSMA-expressing LNCaP cells, gdT cells conjugated with DUPA were more efficient and more potent at killing target cells than unconjugated gdT cells or gdT cells conjugated with a non-PSMA binding small molecule (biotin) at all tested Effector:Target (ET) ratios. In FIG. 13A, the maximum level of cytolysis by DUPA-conjugated gdT cells (gdT-DUPA-L1 and gdT-DUPA-L2) at a 3:1 ratio with targets was attained very rapidly, within two hours after the addition of effectors to the effector cell culture (corresponding to time =25 h in FIG. 13A). In contrast, the non-conjugated gdT cells (gdT) or biotin-conjugated gdT cells (gdT-biotin) at the same ET ratio had a significant delay in their cytolytic effects and did not reach the maximum cytolysis exhibited by the DUPA-conjugated cells even after 72 hours into the assay. This delayed response is demonstrated clearly in FIG. 14A, a bar chart that provides the percent cytolysis 2 hours after addition of the effector cells. A dose response is also evident from the results of testing different Effector:Target (ET) ratios. FIGS. 14A and 14B show that both L1 DUPA and L2 DUPA-conjugated gdT cells killed at least 80% of target cells at 2 h at the 3:1 ET ratio and have reached almost 100% killing by 6 h post treatment, whereas unconjugated gdT cells (gdT) and gdT cells conjugated with non-PSMA targeting compound (gdT-biotin) behaved similarly to one another and had only killed about 14% and —11% target cells at 2 h post treatment, respectively, and between about 40% and 50% target cells at 6 h post treatment. The increased cell killing of the DUPA conjugates was maintained throughout the assay, until at least 72 h (FIGS. 14A-C).

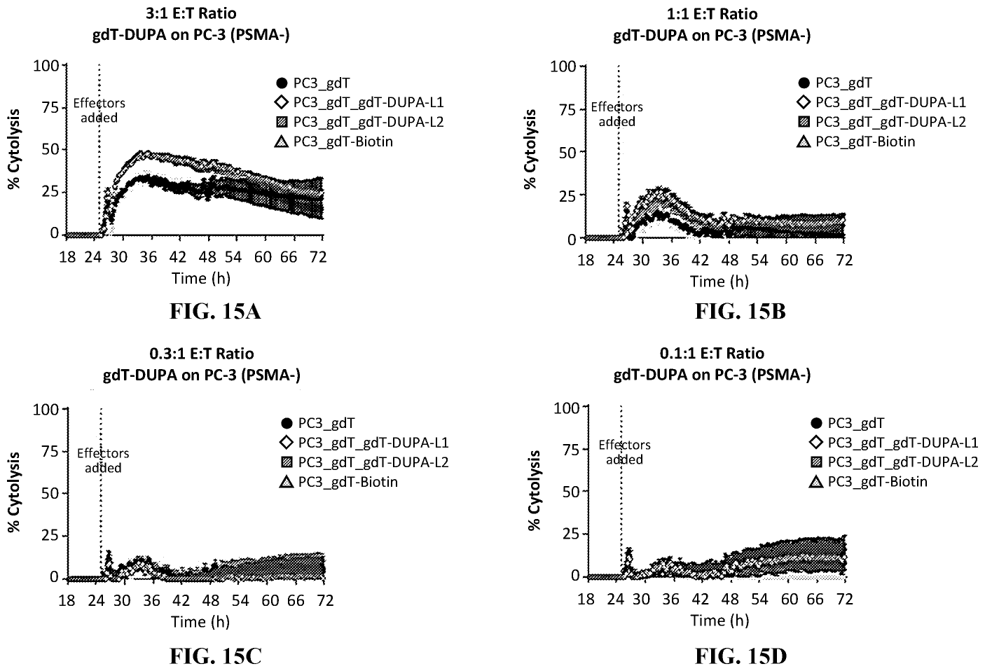

The gap between the higher cell killing capabilities of gdT cells conjugated with DUPA and the lower killing capabilities of gdT cells not conjugated with DUPA (unconjugated gdT cells and gdT cells conjugated with non-PSMA targeting small molecule) was even more pronounced at ET ratios lower than 3:1. At lower Effector:Target ratios, both L1 and L2 DUPA-conjugated cells remained highly potent with maximum cytolysis at greater than 95% and 80% for 1:1 and 0.3:1 ET ratios, respectively, compared to the maximum cell killing achieved by both gdT cells and gdT-biotin cells, which behave almost identically, at 45-48% (1:1 ET ratio) and 19% (0.3:1 ET ratio) (FIGS. 14B and 14C). In addition, the cytolysis displayed by both unconjugated gdT cells and biotin-conjugated gdT cells showed a delayed effect with respect to that of DPA-conjugated gdT cells. In contrast to the results of the 3:1 ET ratio, both the DUPA-conjugated cells at 1:1 and 0.3:1 ET ratios continued to show increasing activity from 2 h to 6 h post treatment but no large increases in cytolysis after 6 h (FIG. 15B and 15C). Even at the lowest ET ratio tested at 0.1:1, both L1 and L2 DUPA-conjugated cells killed at least 40% of the target cells, whereas only minimal cell killing was observed for the gdT cells and gdT-biotin cells, averaging 6% and 13%, respectively (FIG. 14D).

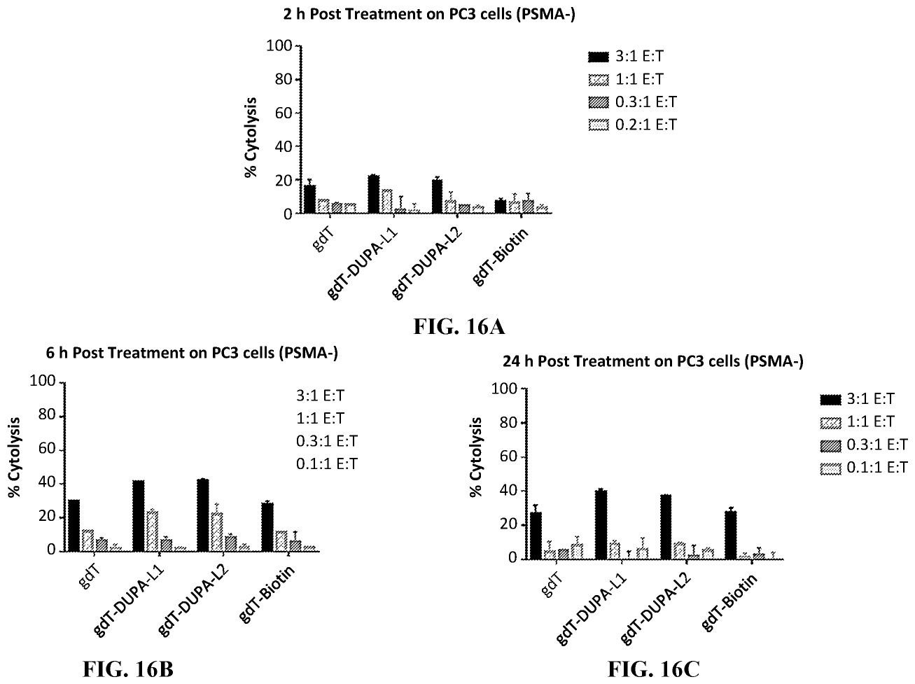

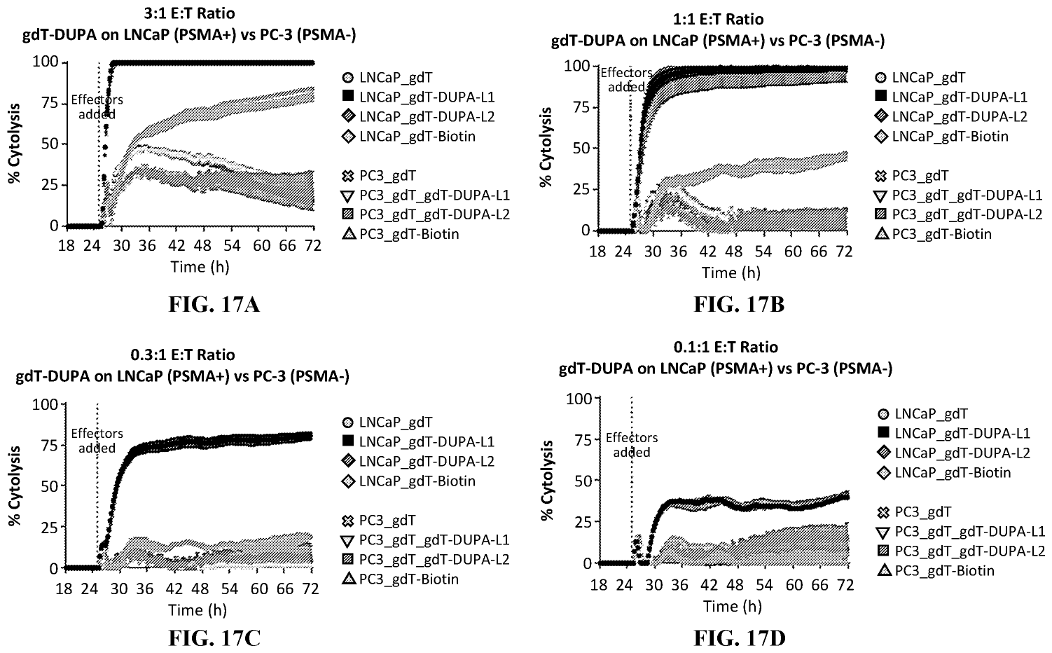

FIG. 16 (A-D) provides the results of the control assays in which the target cells were PSMA-negative PC3 tumor cells. A slight cell killing effect that gradually diminished was observed in both the conjugated and unconjugated gdT cells, particularly at the 3:1 ET ratio (FIG. 16A), presumably due to an inherent cell killing mechanism of gdT cells that targets cancer cells. The slightly higher killing observed with the DUPA-conjugated cells over the control gdT cells and gdT-biotin cells at the 3:1 ET ratio may be due to the presence of low PSMA expression on the cell surface (FIG. 17A-C). Nonetheless, both L1 and L2 DUPA-conjugated gdT cells demonstrated high selectivity towards the PSMA-positive cancer cell line.

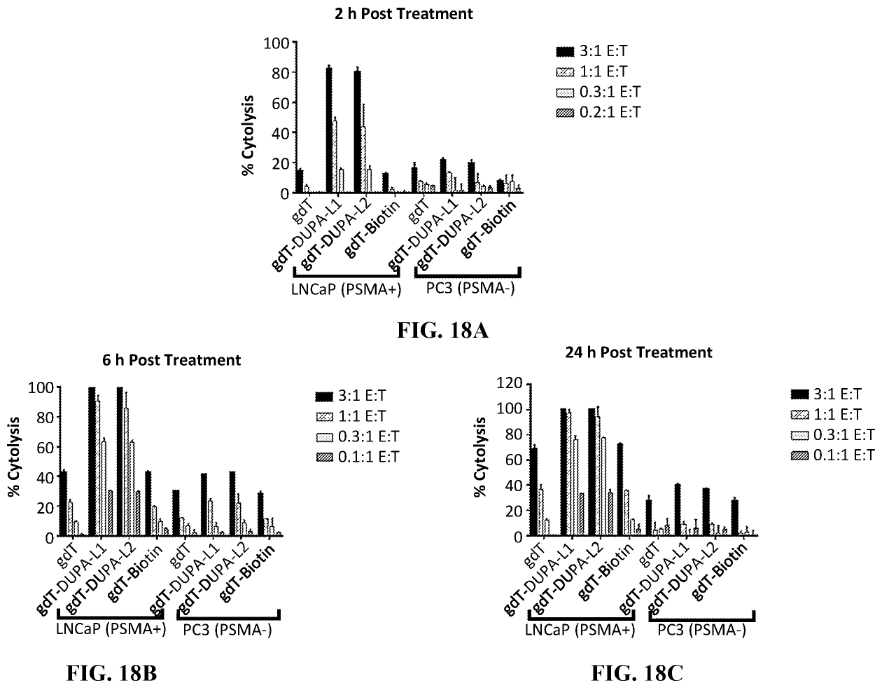

FIG. 18A-D provides the results of cell impedance-based cytotoxicity assays performed essentially as detailed above, in which some of the wells of the assay plate included PSMA-negative PC3 tumor cells as targets and others included PSMA-positive LNCaP tumor cells as targets. At all ET ratios, the cytotoxicity of L1 and L2 DUPA-conjugated gdT cells toward LNCaP tumor cells far exceeded their toxicity toward PC3 tumor cells. The slightly higher killing observed with the DUPA-conjugated cells over the control gdT cells and gdT-biotin cells at 3:1 and 1:1 ET ratios may be due to the presence of low PSMA expression on the PC3 cell surface (FIG. 18A and B). FIG. 19 (A-C) shows in bar graphs the high degree of selectivity of DUPA-conjugated gdT cells toward PSMA-positive tumor cells at all tested E:T ratios. Cells equipped with DUPA demonstrated enhanced cell killing toward LNCaP cells due to the known affinity of DUPA towards PSMA. The low-level cell killing effect observed for the negative control conjugate, gdT-biotin can most likely be attributed to the native tumor cell killing ability of gdT cells. Nevertheless, a high degree of specificity for cytotoxicity toward

PSMA-expressing tumor was observed for the gdT cells conjugated with DUPA and the cells were highly effective at killing PSMA-positive tumor cells at ratios at or below 1:1.

Nonetheless, both the DUPA-conjugated gdT cells have demonstrated high selectivity towards the PSMA positive cancer cell line (FIGS. 7 and 8). Cells equipped with DUPA have shown enhanced cell killing ability towards LNCaP cells due to the known affinity of DUPA towards PSMA. Note that only low cell killing effect (which is most likely attributed to the cell killing ability of gdT cells by itself) was observed for the negative control conjugate, gdT-Biotin, (biotin does not bind to PSMA). Taken together, it appears that in all the assays the gdT-DUPA conjugates are most efficient between ET ratios of 0.3-1:1 in which the cell killing is maintained at >80% while maintaining a low non-specific cell killing.

1. A gamma delta T (gdT) cell, Natural Killer (NK) cell, or macrophage comprising 2-[3-(1,3-dicarboxypropyl)ureido]pentanedioic acid (DUPA) conjugated to the cell surface.

2. A cell according to claim 1, wherein DUPA is conjugated to the surface of the cell via a linker.

3. A cell according to claim 2, wherein the linker comprises a functional group that reacts with lysine.

4. A cell according to claim 3, wherein the functional group is N-hydroxysuccinimide (NHS), pentafluorophenyl, or p-nitrophenyl.

5. A cell according to claim 2, wherein the linker comprises one or more of (CH₂)ₙ-, —(CH₂CH₂O)ₙ-, —CH₂(C═O)—, —(C═O)—, —(C═O)CH₂CH₂OCH₂CH₂OCH₂CH₂—, —(C═O)CH₂CH₂O(CH₂CH₂O)ₙCH₂CH₂NH—, —(C═O)CH₂CH₂(C═O)—, —(C═O)CH₂CH₂O(CH₂CH₂O)ₙCH₂CH₂NH(C═O)—, an amino acid, a dipeptide, a tripeptide, polyglycine, p-aminobenzyl (PAB), piperazine, piperidine, or a triazole, where n can be, independently, 1-30.

6. The innate immune system cell of claim 5, wherein the linker comprises (CH₂)ₙ-, —(CH₂CH₂O)ₙ-, or —CH₂(C═O)—.

7. A cell according to any of claims 1-6, wherein the cell is a gamma delta T (gdT) cell.

8. The gdT cell of claim 7, wherein the gdT cell is from a cell line.

9. The gdT cell of claim 8, wherein the gdT cell is irradiated.

10. The gdT cell of claim 7, wherein the gdT cell is a primary cell.

11. The gdT cell of claim 10, wherein the gdT cell is isolated from PBMCs or cord blood.

12. A population of DUPA-conjugated gamma delta T cells comprising a plurality of cells of claim 7.

13. A pharmaceutical composition comprising a population of gamma delta T cells according to claim 12.

14. The pharmaceutical composition of claim 13, wherein the cells are provided in a bag, vial, or tube, wherein the cells are optionally frozen.

15. A method of treating a subject having a PSMA-positive cancer, comprising administering one or more doses of an effective amount of the population of gamma delta T cells of claim 12 to the subject.

16. A method according to claim 15, wherein the PSMA-positive cancer is prostate cancer.

17. A method according to claim 15, wherein the population of cells is administered by injection or infusion.

18. A method according to claim 15, wherein more than one dose is administered.

19. A population of DUPA-conjugated gamma delta T cells according to claim 12 for use in a method of treating a subject having a PSMA-positive cancer, wherein the subject is administered one or more doses of an effective amount of the gamma delta T cells.

20. A population of DUPA-conjugated gamma delta T cells according to claim 19, wherein the PSMA-positive cancer is prostate cancer.

21. A population of DUPA-conjugated gamma delta T cells according to claim 19, wherein the population of cells is administered by injection or infusion.

22. A population of DUPA-conjugated gamma delta T cells according to claim 19, wherein the population of cells is administered more than once.

23. A cell according to any of claims 1-6, wherein the cell is a Natural Killer (NK) cell.

24. The NK cell of claim 23, wherein the NK cell is a primary cell.

25. The NK cell of claim 24, wherein the NK cell is isolated from PBMCs or cord blood.

26. The NK cell of claim 23, wherein the NK cell is from a cell line.

27. The NK cell of claim 23, wherein the NK cell is irradiated.

28. The NK cell of claim 26, wherein the NK cell is a KHYG cell.

29. A population of DUPA-conjugated NK cells comprising a plurality of cells of claim 23.

30. A pharmaceutical composition comprising a population of NK cells according to claim 29.

31. The pharmaceutical composition of claim 30, wherein the cells are provided in a bag, vial, or tube, wherein the cells are optionally frozen.

32. A method of treating a subject having a PSMA-positive cancer, comprising administering one or more doses of an effective amount of the population of NK cells of claim 29 or a pharmaceutical composition thereof to the subject.

33. A method according to claim 32, wherein the PSMA-positive cancer is prostate cancer.

34. A method according to claim 32, wherein the population of cells is administered by injection or infusion.

35. A method according to claim 32, wherein more than one dose is administered.

36. A population of DUPA-conjugated NK cells according to claim 29 for use in a method of treating a subject having a PSMA-positive cancer, wherein the subject is administered one or more doses of an effective amount of the NK cells.

37. A population of DUPA-conjugated NK cells according to claim 36, wherein the PSMA-positive cancer is prostate cancer.

38. A population of DUPA-conjugated gamma delta T cells according to claim 35, wherein the population of cells is administered by injection or infusion.

39. A population of DUPA-conjugated gamma delta T cells according to claim 35, wherein the population of cells is administered more than once.

40. A cell according to any of claims 1-6, wherein the cell is a macrophage.

41. The macrophage of claim 40, wherein the macrophage is from a cell line.

42. The macrophage of claim 40, wherein the macrophage is a primary cell.

43. The macrophage of claim 42, wherein the macrophage is isolated from PBMCs.

44. A population of DUPA-conjugated macrophages comprising a plurality of cells of claim 40.

45. A pharmaceutical composition comprising a population of macrophages according to claim

44.

46. The pharmaceutical composition of claim 45, wherein the macrophages are provided in a bag, vial, or tube, wherein the cells are optionally frozen.

47. A method of treating a subject having a PSMA-positive cancer, comprising administering one or more doses of an effective amount of the population of macrophages of claim 44 or a pharmaceutical composition thereof to the subject.

48. A method according to claim 47, wherein the PSMA-positive cancer is prostate cancer.

49. A method according to claim 47, wherein the population of macrophages is administered by injection or infusion.

50. A method according to claim 47, wherein more than one dose is administered.

51. A population of DUPA-conjugated macrophages according to claim 50 or a pharmaceutical composition thereof, for use in a method of treating a subject having a PSMA-positive cancer, wherein the subject is administered one or more doses of an effective amount of the macrophages.

52. A population of DUPA-conjugated macrophages or pharmaceutical composition according to claim 51, wherein the PSMA-positive cancer is prostate cancer.

53. A population of DUPA-conjugated macrophages or pharmaceutical composition according to claim 51, wherein the population of macrophages is administered by injection or infusion.

54. A population of DUPA-conjugated macrophages or pharmaceutical composition according to claim 51, wherein the population of macrophages is administered more than once.
